(19) United States
(12) Patent Application Publication
(10) Pub. No.: US 2024/0325001 A1
(43) Pub. Date: Oct. 3, 2024

(54) WEARABLE APTAMER NANOBIOSENSOR FOR NON-INVASIVE FEMALE HORMONE MONITORING

(71) Applicant: California Institute of Technology, Pasadena, CA (US)

(72) Inventors: Wei GAO, San Marino, CA (US); Cui YE, Pasadena, CA (US)

(21) Appl. No.: 18/605,727

(22) Filed: Mar. 14, 2024

Related U.S. Application Data
(60) Provisional application No. 63/452,099, filed on Mar. 14, 2023.

(51) Int. Cl. A61B 10/00; A61B 90/00

(52) U.S. Cl. CPC A61B 10/0064; A61B 90/39

(57) ABSTRACT

Some implementations of the disclosure relate to a wearable biosensor device including: a microfluidic module configured to collect a sweat sample from skin of a user, route the sweat sample to a sensing reservoir that is filled with the sweat sample, and route additional sweat away from the sensing reservoir when the sensing reservoir is filled; and a sensor assembly configured to quantify the biomarker of the sweat sample in the sensing reservoir to determine a concentration of the biomarker present in the sweat sample. The sensor assembly includes a biorecognition interface having a surface functionalized with an aptamer that binds to the biomarker.

### CROSS-REFERENCE TO RELATED APPLICATIONS

This application claims the benefit of U.S. Provisional Patent Application No. 63/452,099, filed Mar. 14, 2023, and titled “A Wearable Aptamer Nanobiosensor For Non-Invasive Female Hormone Monitoring”, is incorporated herein by reference in its entirety.

This invention was made with government support under Grant No. HL155815 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Jun. 5, 2024, is named 30KJ-383124-US_SL.xml and is 6,362 bytes in size.

### TECHNICAL FIELD

The present disclosure relates generally to systems and methods for biomarker monitoring using a wearable biosensor device. Particular implementations are directed to automatic and non-invasive monitoring of protein or hormone biomarkers using a wearable aptamer biosensor that collects sweat samples.

### BACKGROUND

Recent advances in flexible electronics and digital health have transformed conventional laboratory tests into remote wearable molecular sensing that enables real-time monitoring of physiological biomarkers. Sweat contains abundant biochemical molecules such as electrolytes, metabolites, proteins, and steroid hormones, and importantly, it is readily accessible by non-invasive techniques. However, currently reported wearable biosensors are often restricted to the detection of a limited selection of biomarkers at μM or greater concentrations via ion-selective and enzymatic sensors or direct oxidation/reduction.

For example, monitoring of female hormones such as estradiol (“E2”), also spelled “oestradiol”, is of great interest in fertility and women's health. However, existing approaches for measuring estradiol typically require invasive blood draws and/or bulky laboratory equipment, and they have a low detection throughput for routine clinical use. Although there are commercially available “at-home” estrogen test kits based on finger-prick blood drop, saliva, or 24-hour urine, all of them require the collected samples to be sent out for lengthy laboratory analysis. Despite the high demand, estradiol cannot be detected with existing wearable sensing strategies such as enzymatic or ion-selective electrodes. Reagentless wearable in situ monitoring of female hormones such as estradiol in sweat is highly challenging due to the extremely low concentration (pM level) and interpersonal/intrapersonal variations in sweat accessibility. Commercial point-of-care biomarker monitors are still bulky in size and cannot reach the picomolar-level sensitivity needed to assess hormone levels.

### SUMMARY

The technology described herein relates to wearable aptamer sensor systems and methods capable of automatic, reagentless, and real-time monitoring of low levels of biomarkers such as steroid hormone and protein biomarkers.

In one embodiment, a wearable biosensor device comprises: a microfluidic module configured to collect a sweat sample from skin of a user, route the sweat sample to a sensing reservoir, and route additional sweat away from the sensing reservoir when the sensing reservoir is filled; and a sensor assembly configured to quantify a biomarker contained in the sweat sample in the sensing reservoir to determine a concentration of the biomarker present in the sweat sample, the sensor assembly comprising a biorecognition interface having a surface functionalized with an aptamer that binds to the biomarker.

In some implementations, the wearable biosensor device further comprises an iontophoresis module that stimulates production of the sweat sample.

In some implementations, the microfluidic module comprises: an inlet for collecting the sweat sample; the sensing reservoir; an outlet for providing an outflow of the additional sweat, the outlet fluidically positioned between the inlet and the sensing reservoir; and a first microvalve fluidically positioned between the inlet and the outlet, the first microvalve configured to change from a closed state to an open state after the sensing reservoir is filled.

In some implementations, the first microvalve is a first capillary bursting valve (CBV), the first CBV having a burst pressure (BP) that is configured to be exceeded after the sensing reservoir is filled.

In some implementations, the microfluidic module further comprises a second CBV, the second CBV fluidically positioned after the sensing reservoir; and the second CBV is configured to be in a closed state after the sensing reservoir is filled.

In some implementations, the sensor assembly further comprises a working electrode; the surface of the biorecognition interface is further functionalized with a labeled molecule containing an electroactive label; in response to the aptamer binding to the biomarker, the biorecognition interface is configured to release the labeled molecule; and a surface of the working electrode is functionalized to bind to the labeled molecule released from the biorecognition interface.

In some implementations, the aptamer comprises first single-stranded deoxyribonucleic acid (ssDNA) selective to the biomarker; the labeled molecule comprises second ssDNA; and the surface of the working electrode is functionalized with third ssDNA configured to hybridize to the second ssDNA.

In some implementations, the first ssDNA is hybridized to the second ssDNA as a partially hybridized sequence; and in the presence of the biomarker, the first ssDNA is configured to release the second ssDNA from the surface of the biorecognition interface due to a higher affinity of the first ssDNA to the biomarker than the partially hybridized sequence.

In some implementations, the sensor assembly further comprises a counter electrode; and the wearable biosensor device is configured to apply an electric field between the counter electrode and the working electrode to promote transport of the released molecule containing the electroactive label to the working electrode.

In some implementations, the wearable biosensor device further comprises a flexible printed circuit board (FPCB) electrically coupled to the sensor assembly, the FPCB configured to apply the electric field as a bias potential between the counter electrode and the working electrode.

In some implementations, the surface of the working electrode comprises: one or more layers of gold nanoparticles (AuNPs); and one or more layers of MXene formed over the one or more layers of AuNPs.

In some implementations, the biomarker is a reproductive hormone; and the sensor assembly is configured to quantify the reproductive hormone to determine the concentration of the reproductive hormone with a sensitivity of 1 picomole or less.

In some implementations, the surface of the biorecognition interface faces the surface of the working electrode; the surface of the biorecognition interface forms a first wall of the sensing reservoir; and the surface of the working electrode forms a second wall of the sensing reservoir, opposite the first wall.

In some implementations, the sensor assembly further comprises: an ionic strength sensor configured to measure an ionic strength of the sweat sample; and a pH sensor configured to measure a pH level of the sweat sample, wherein the wearable biosensor device is configured to calibrate readings from the working electrode based on measurements made by the ionic strength sensor and the pH sensor.

In some implementations, the wearable biosensor device comprises: a disposable patch including an iontophoresis module that stimulates production of the sweat sample, the microfluidic module, and the sensor assembly, the disposable patch comprising an adhesive to directly adhere the disposable patch to the skin; and a FPCB coupled to the disposable patch, the FPCB configured to receive signals from the sensor assembly and power the wearable biosensor device, wherein the FPCB is configured to be worn around a finger of the user.

In some implementations, the biomarker is a first type of biomarker; the sweat sample further comprises a second type of biomarker different from the first type of biomarker; the surface of the biorecognition interface is further functionalized with a second aptamer that binds to the second type of biomarker; and the sensor assembly is further configured to quantify the second type of biomarker of the sweat sample in the sensing reservoir to determine a concentration of the second type of biomarker present in the sweat sample.

In some implementations, the first type of biomarker is a first type of female reproductive hormone; and the second type of biomarker is a second type of female reproductive hormone.

In one embodiment, a method comprises: receiving, via an inlet of a microfluidic module of a wearable biosensor device, a sweat sample collected from skin, the sweat sample including a biomarker; collecting, within a sensing reservoir of the microfluidic module, the sweat sample, the microfluidic module comprising an outlet fluidically positioned between the inlet and the sensing reservoir, and a microvalve fluidically positioned between the inlet and the outlet, the microvalve configured to change from a closed state to an open state after the sensing reservoir is filled; when the sensing reservoir is filled with the sweat sample, releasing, via the outlet, an additional sweat sample received by the microfluidic module via the inlet; and estimating, using an aptamer sensor assembly of the wearable biosensor device, a concentration of the biomarker in the sweat sample collected in the sensing reservoir.

In some implementations, the method further comprises: regenerating the aptamer sensor assembly by rinsing the microfluidic module with deionized water or a solution with low ionic strength or acidic pH.

In some implementations, estimating the concentration of the biomarker in the sweat sample collected in the sensing reservoir comprises: binding, within the sensing reservoir, the biomarker of the sweat sample to an aptamer on a surface of a biorecognition interface of the aptamer sensor assembly; in response to binding the biomarker to the aptamer, releasing, from the surface of the biorecognition interface, a molecule containing an electroactive label; collecting, at a surface of a working electrode of the aptamer sensor assembly, the molecule released from the surface of the biorecognition interface; and measuring an amount of electroactive label present at the surface of the working electrode to estimate a concentration of the biomarker present in the sweat sample.

In some implementations, estimating the concentration of the biomarker in the sweat sample collected in the sensing reservoir, further comprises: applying an electric field between a counter electrode of the aptamer sensor assembly and the working electrode to promote transport to the working electrode of the molecule released from the surface of the biorecognition interface.

In one embodiment, a method comprises: adhering, to skin of a user, a patch that includes a microfluidic module and sensor assembly; collecting, in a sensing reservoir of the microfluidic module, a sweat sample obtained from the skin, the sweat sample including a reproductive hormone biomarker; and automatically estimating, using an aptamer sensor assembly in the sensing reservoir, a concentration of the reproductive hormone biomarker in the sweat sample.

In some implementations, the method further comprises: presenting to the user, in real-time, via a mobile device communicatively coupled to the patch via a wireless communication medium, the concentration of the reproductive hormone biomarker estimated using the aptamer sensor assembly.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and case of illustration, the elements in the figures have not necessarily been drawn to scale.

FIG. 1A illustrates an example biosensor device, and an environment for using the biosensor device, in accordance with some implementations of the disclosure.

FIG. 1B shows an exploded view of the biosensor device of FIG. 1A.

FIG. 1C is a schematic diagram illustrating components of an assembled aptamer sensor patch of a biosensor device, in accordance with some implementations of the disclosure.

FIG. 1D is a schematic diagram showing a structure and valve operation of a microfluidic module of an assembled aptamer sensor patch, in accordance with some implementations of the disclosure.

FIG. 2 is a schematic diagram conceptually illustrating a particular embodiment of the operation of an aptamer sensor assembly that is configured to provide the reagentless in situ quantification of the biomarker estradiol, in accordance with the disclosure.

FIG. 3A shows a photograph of a flatted electronic system of a particular embodiment of a biosensor device, in accordance with some implementations of the disclosure.

FIG. 3B shows a corresponding block diagram of the electronic system of FIG. 3A.

FIG. 4 is a schematic diagram illustrating an example method of assembling a aptamer sensor patch, in accordance with some implementations of the disclosure.

FIG. 5 is a schematic diagram illustrating an example method of inkjet printing components of a sensor assembly, and corresponding scanning electron microscope characterizations of printed nanostructure patterns at different stages of inkjet printing, in accordance with a particular embodiment of the technology described herein.

FIG. 6 is an operational flow diagram example operations performed in a microfluidic module of aptamer sensor device during automatic and controlled biofluid sampling, in accordance with some implementations of the disclosure.

FIG. 7 is a schematic diagram illustrating different stages of biofluid sampling in a particular microfluidic valving design for realizing a precise sampling of a biofluid sample for biomarker detection, in accordance with some implementations of the disclosure.

FIG. 8 is an operational flow diagram illustrating example operations performed by an aptamer sensor assembly of a biosensor device during reagentless, automatic, and in-situ recognition and analysis of biomarkers contained in a biofluid sample, in accordance with some implementations of the disclosure.

FIG. 9 is a schematic diagram illustrating the operation of an aptamer sensor assembly that is configured to provide the reagentless in situ quantification of the biomarker estradiol using a biorecognition interface modified with an estradiol-selective DNA aptamer facing a AuNPs-MXene-based detection working electrode (WE) modified with capture ssDNA, in accordance with a particular embodiment of the disclosure.

FIG. 10 illustrates an example graphical user interface (GUI) that can be presented to a user by running a mobile application used in conjunction with a wearable biosensor device for noninvasive, automatic reproductive hormone monitoring, in accordance with some implementations of the disclosure.

FIG. 11A shows a photograph of a particular embodiment of sensor patch that is disposable, in accordance with some implementations of the disclosure.

FIG. 11B shows a photograph of a particular embodiment of a biosensor device that is implemented as a fully integrated wireless wearable patch worn on a finger for hormone monitoring, in accordance with some implementations of the disclosure.

FIG. 12 illustrates non-invasive monitoring of estradiol realized through sweat analysis using a skin-interfaced wearable sensor, in accordance with some implementations of the disclosure.

FIG. 13 illustrates hormonal fluctuations over a menstrual cycle.

FIG. 14A illustrates results of continuous female hormone monitoring for two menstrual cycles for a first female participant, including plots of urine luteinizing hormone (LH) levels tested by a commercial LH strips kit, estradiol levels in serum tested by an enzyme-linked immunosorbent assay (ELISA) sensor, and estradiol levels in sweat tested by an aptamer sensor in accordance with some implementations.

FIG. 14B illustrates results of continuous female hormone monitoring for two menstrual cycles for a second female participant, including plots of urine LH levels tested by a commercial LH strips kit, estradiol levels in serum tested by an ELISA sensor, and estradiol levels in sweat tested by an aptamer sensor in accordance with some implementations.

FIG. 14C shows a plot of a measured correlation of estradiol levels between human sweat and serum, in accordance with some implementations of the disclosure.

FIG. 14D shows plots corresponding to results of on-body multiplexed physicochemical sensing and estradiol quantification with real-time sensor calibrations in the follicular phase of the menstrual cycle for three human participants, using a wearable aptamer sensor in accordance with some implementation of the disclosure.

FIG. 14E shows plots corresponding to results of on-body multiplexed physicochemical sensing and estradiol quantification with real-time sensor calibrations in the ovulation phase of the menstrual cycle for three human participants, using a wearable aptamer sensor in accordance with some implementation of the disclosure.

FIG. 14F shows plots corresponding to results of on-body multiplexed physicochemical sensing and estradiol quantification with real-time sensor calibrations in the luteal phase of the menstrual cycle for three human participants, using a wearable aptamer sensor in accordance with some implementation of the disclosure.

FIG. 15 shows a schematic of a reagentless electrochemical aptamer sensor based on competitive redox probe displacement and recapture, in accordance with some implementations of the disclosure.

FIG. 16 shows an aptamer and DNA sequences used in accordance with some implementations of the disclosure. Figure discloses SEQ ID NOS 2-4, respectively, in order of appearance.

FIG. 17A shows a schematic of a surface modification procedure of a biorecognition interface, in accordance with some implementations of the disclosure.

FIG. 17B shows a schematic of a surface modification procedure of a detection working electrode, in accordance with some implementations of the disclosure.

FIG. 17C shows a scanning electron microscope (SEM) image of an inkjet-printed AuNPs-MXene electrode, in accordance with some implementations of the disclosure.

FIG. 17D shows a transmission electron microscopy characterization of AuNP-MXene, in accordance with some implementations of the disclosure.

FIG. 17E shows a plot corresponding to a differential pulse voltammetry (DPV) characterization of a working electrode in 0.1×PBS (pH 7.4) containing 2.0 mM K₄Fe(CN)₆/K₃Fe(CN)₆ (1:1) after each surface-modification step: AuNPs, AuNPs-MXene, SH-ssDNA and 6-mercapto-1-hexanol (MCH), in accordance with some implementations of the disclosure.

FIG. 17F shows a plot corresponding to an OCP-EIS characterization of a working electrode in 0.1×PBS (pH 7.4) containing 2.0 mM K₄Fe(CN)₆/K₃Fe(CN)₆ (1:1) after each surface-modification step: AuNPs, AuNPs-MXene, SH-ssDNA and MCH, in accordance with some implementations of the disclosure.

FIG. 17G shows a plot corresponding to a square wave voltammogram (SWV) response of a modified AuNPs-MXene-based sensor before and after incubation with 50 PM of estradiol, in accordance with some implementations of the disclosure.

FIG. 17H shows a plot corresponding to a comparison of the detection performance of working electrode substrates including Au, AuNPs, AuNPs-MXene and AuNPs-MXcnc-MPA.

FIG. 17I shows a plot corresponding to the SWV response of aptamer estradiol sensors in artificial sweat (0.2×PBS, pH 7.4) with 0, 0.1, 0.5, 1, 5, 10, 50 and 100 pM of estradiol, in accordance with some implementations of the disclosure.

FIG. 17J shows a calibration plot corresponding to the SWV response of FIG. 17I, based on the peak current height of the SWV voltammograms, where each SWV voltammogram was obtained from an independent estradiol sensor, in accordance with some implementations of the disclosure.

FIG. 17K shows selectivity of an aptamer sensor to potential interferences (50 pM) in human sweat, in accordance with some implementations of the disclosure.

FIG. 17L shows validation of an aptamer sensor towards estradiol quantification in iontophoresis-induced human sweat samples with ELISA, in accordance with some implementations of the disclosure.

FIG. 17M shows calibration plots of aptamer sensors with 10 minutes of incubation measured with and without the assistance of an external electric field, in accordance with some implementations of the disclosure.

FIG. 17N shows experimental and simulation results of a sensor response with and without the assistance of an external electric field after 10 minutes of incubation, in accordance with some implementations of the disclosure.

FIG. 17O shows a plot corresponding to SWV voltammograms showing the regeneration and repetitive use of aptamer estradiol sensors with 50 pM of estradiol, in accordance with some implementations of the disclosure.

FIG. 17P shows a plot of the corresponding peak current height of the SWV voltammograms of FIG. 17O.

FIG. 18A shows optical images showing phases of CBV-modulated in situ sweat sampling process towards automatic estradiol analysis, in accordance with some implementations of the disclosure.

FIG. 18B shows a plot corresponding to the SWV voltammograms of aptamer estradiol sensors in a laboratory microfluidic flow test using artificial sweat (0.2×PBS, pH 7.4) containing 0, 1, 5, 10, 20, 40, 60, 80 and 100 pM of estradiol, in accordance with some implementations of the disclosure.

FIG. 18C shows a calibration plot corresponding to the SWV voltammograms of FIG. 18B, based on the peak current height of the SWV voltammograms, in accordance with some implementations of the disclosure.

FIG. 18D shows a potentiometric response and corresponding calibration plot (inset) of pH sensors in artificial sweat (0.2×PBS), in accordance with some implementations of the disclosure.

FIG. 18E shows an impedimetric response and the corresponding calibration plot (inset) of ionic strength sensors in artificial sweat (0.2×PBS, pH 7.4), in accordance with some implementations of the disclosure.

The figures are not exhaustive and do not limit the present disclosure to the precise form disclosed.

### DETAILED DESCRIPTION

Despite recent efforts in the development of wearable biosensors for trace-level biomarkers, the accurate and in situ detection of biomarkers such as sweat protein or steroid hormone biomarkers remains a major challenge due to their extremely low concentrations (e.g., nM or pM levels) and the large interpersonal and intrapersonal variations in sweat compositions. For example, although bioaffinity receptors have been integrated for the detection of protein biomarkers, such techniques typically require lengthy target incubation, labor-intensive washing steps, and the addition of redox solutions for signal transduction. As such, there is a need for a wearable biosensing technology that allows automatic in situ monitoring of ultra-low level circulating biomarkers at home and in community settings. Highly sensitive and accurate on-site quantification of protein or steroid hormone levels (e.g. female hormones) in noninvasively accessible biological fluids would be highly desired to ensure optimal medical decision-making at the individual level.

To this end, some implementations of disclosure are directed to aptamer-based systems and methods for wearable and real-time electrochemical detection of low-concentration protein and steroid hormone biomarkers such as female hormones in sweat. In accordance with some implementations of the disclosure, a biosensor device for biomarker sampling and sensing can include: an iontophoresis module that stimulates production of sweat, a microfluidic module for controlled sweat sampling using one or more microvalves (e.g., CBVs), and a reagentless, electrochemical aptamer sensor assembly for quantifying a biomarker contained in the sweat. The electrochemical aptamer sensor assembly can contain a biorecognition interface including aptamers that bind to the biomarkers, and a working electrode configured to measure an amount of electroactive label molecules released from the biorecognition interface when the biomarkers bind to the aptamers. Based on the measured amount of electroactive label molecules, a concentration of the biomarkers in the sweat sample can be estimated. In particular implementations, the biorecognition interface of the aptamer sensor assembly functions as a strand-displacement aptamer switch. In accordance with some particular implementations, the aptamer sensory assembly can be part of a sensor array that also includes sensors for ionic strength, pH, and/or temperature measurements, for the real-calibration of the aptamer sensor.

Various benefits can be realized by implementing the systems and methods described herein. First, the wearable biosensor device described herein can enable real-time, non-invasive, and wireless biomarker analysis of very low concentration biomarkers in all populations. This could be particularly suitable for enabling convenient, at-home monitoring of reproductive hormones or other trace-label biomarkers present in sweat of a user toward a wide variety of personalized medicine applications. Second, by virtue incorporating precise microfluidic sweat sampling controlled by microvalves, in combination with particular nanomaterials and chemistry techniques with the aptamer sensor assembly, the technology described herein could realize sweat estradiol or other biomarker analysis with high sensitivity, selectivity, and efficiency. For example, the technology described herein could be used to realize highly sensitive detection of ultra-low-level biomarkers in situ with a subpicomolar level of sensitivity. Furthermore, experiments further described herein validated the technology for use in monitoring female hormone levels, including data that identified a high correlation of estradiol levels between sweat and blood, and measured a cyclical fluctuation in sweat estradiol during menstrual cycles.

These and other benefits realized by implementing the technology described herein are further describe below.

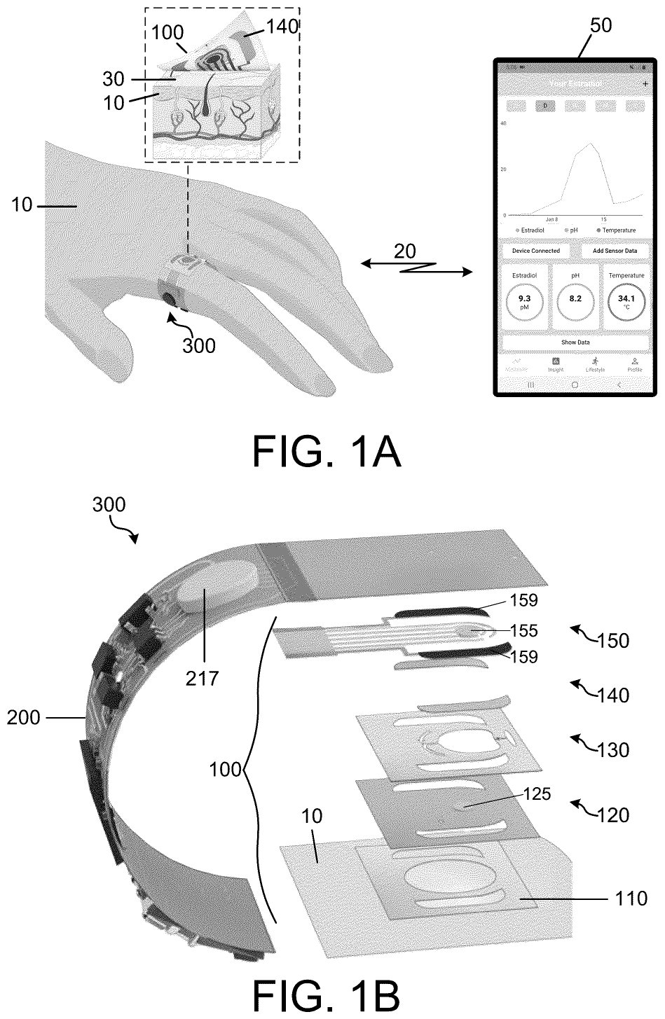

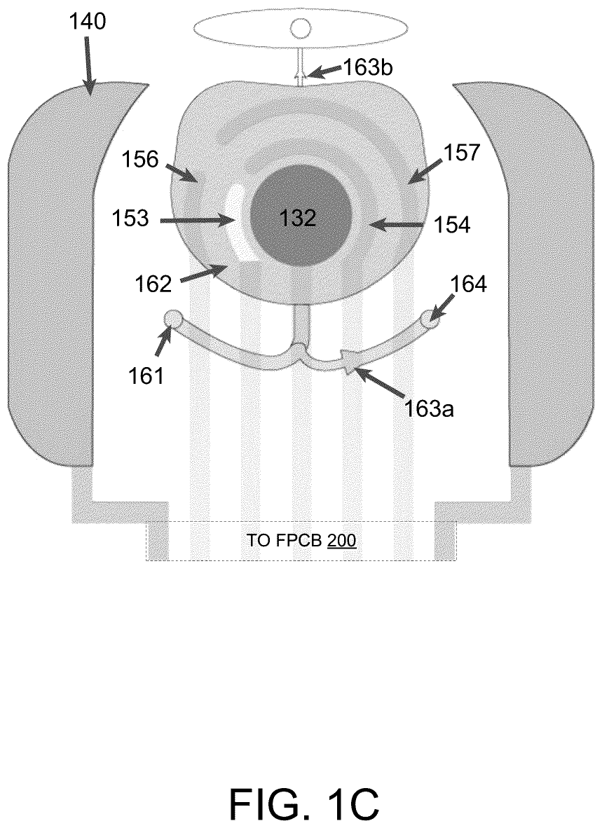

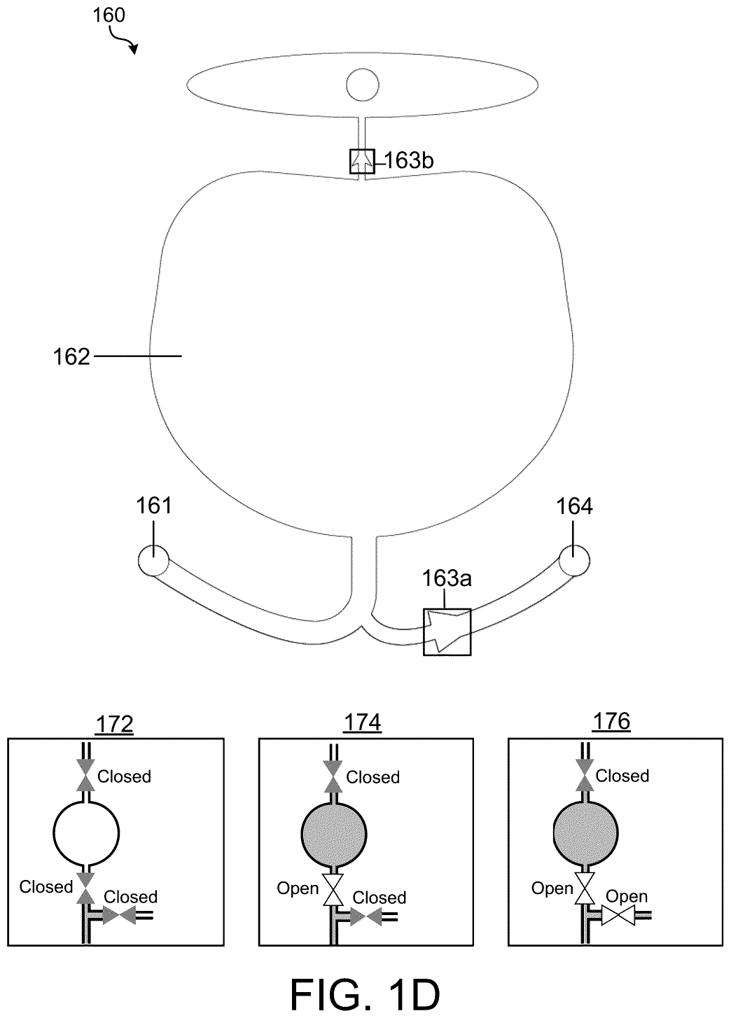

FIGS. 1A-1D illustrate an example biosensor device 300, and an environment for using the biosensor device 300, in accordance with some implementations of the disclosure. The biosensor device 300 includes an aptamer sensor patch 100 electrically and mechanically coupled to a FPCB 200. In the illustrated implementations, the aptamer sensor patch 100 can be removably coupled to FPCB 200. In other implementations, an aptamer sensor patch 100 can be permanently affixed to FPCB 200.

The aptamer sensor patch 100 of the biosensor device 300 can be adhered to the skin 10 of a user (e.g., a human patient). As depicted by FIG. 1A, which shows a cross-sectional view of the aptamer sensor patch 100 adhered to skin 10, the aptamer sensor patch 100 can include one or more layers including a medical adhesive (e.g., medical tape) used to directly attach the sensor patch 100 to skin 10. Iontophoresis electrodes 159 of the aptamer sensor patch 100 can interface the skin 10 with a layer of hydrogel agent 140 applied in between to stimulate the production of sweat 30. The hydrogel agent 140, which can be a component of sensor patch 100, can be an agarose gel containing carbachol (carbagel). An electric current can travel to the electrodes 159, which enable the transdermal transport of carbachol to the sweat glands, triggering the flow of the sweat stimulating agent into the skin 10, and stimulating the production of sweat 30 as needed. An iontophoresis module, including the pair of electrodes, can provide the benefit of on-demand delivery of a hydrogel agent (e.g., cholinergic agonist carbachol from the carbagel) for autonomous sweat stimulation throughout daily activities without the need for vigorous exercise.

During operation, the biosensor device 300 is configured to collect biophysical data corresponding to the user, including data associated with biomarkers collected from the user's sweat 30, and communicate the data to a mobile device 50 via a wireless communication link 20. The wireless communication link 20 can be a radio frequency link such as a Bluetooth® or Bluetooth® low energy (LE) link, a Wi-Fi® link, a ZigBee link, or some other suitable wireless communication link. In some embodiments, a low energy and/or short-range wireless communication link can preferably be used for data transfer. The mobile device 50 can be a smartphone, a smartwatch, a head mounted display (HMD), or other suitable mobile device that can run an application that displays health information (e.g., hormone biomarker data, temperature data, etc.) associated with the data received from the biosensor device 300. In some implementations, the application can analyze and/or organize data collected from the biosensor device 300.

FIG. 1B shows an exploded view of biosensor device 300, in accordance with some implementations of the disclosure. The biosensor device 300 includes aptamer sensor patch 100 and FPCB 200. The aptamer sensor patch 100 includes accumulation layer 110, biorecognition interface layer 120, collection/microfluidic channel layer 130, hydrogel agent 140 (e.g. carbagels), and sensor assembly 150. It should be appreciated that FIG. 1B shows one example arrangement for layering components of aptamer sensor patch 100, and that depending on the fabrication process, the aptamer sensor patch 100 could be assembled using fewer layers or additional layers.

The bottom accumulation layer 110 is configured to contact and adhere to skin 10 of a user wearing biosensor device 300. The bottom accumulation layer 110 includes a sweat accumulation chamber. The collection/microfluidic channel layer 130 can include cutouts for components of a microfluidic module of biosensor device 300, including an inlet, outlet, a biofluid detection/sensing reservoir, channels, and/or channel microvalves (e.g., CBVs).

The biorecognition interface layer 120 includes a biorecognition interface 125 that in conjunction with working electrode 155 of sensor assembly 150 functions as an aptamer sensor. The biorecognition interface 120 has a surface including a biomarker-selective aptamer and a molecule including a electroactive label chemical. As further described below, when the sensor patch 100 is exposed to a biofluid (e.g., sweat) containing biomarkers, the biomarkers can competitively bind with the aptamer, resulting in the release of a molecule containing the electroactive label chemical from the biorecognition interface. The released molecule containing the electroactive label chemical can then be captured by working electrode 155 for biomarker estimation. To this end, the surface of working electrode 155 can include capture molecules that bind to the molecule containing the electroactive label. In some implementations, the working electrode 155 can be coated with a nanoparticle surface that absorbs the capture molecules that bind to the biomarker of interest. In particular implementations, the working electrode 121a surface includes AuNPs modified with MXene. Other nanoparticles that could be utilized in other implementations include iron oxide nanoparticles, quantum dots, silver nanoparticles, copper nanoparticles, copper oxide nanoparticles, etc.

In a particular implementation, the biomarker-selective aptamer is partially hybridized with the molecule including the electroactive label chemical. In the presence of the biomarker, the molecule containing the electroactive label can be released because of the higher affinity of the aptamer to the biomarker than the partially hybridized sequence. In a particular implementation, the biorecognition interface 125 is modified with a female hormone selective aptamer. For example, the aptamer can be configured to bind to estradiol or progesterone. In a particular implementation, the aptamer is a DNA. For example, the aptamer can be a ssDNA. In such implementations, the aptamer sensor assembly can utilize a strand-displacement aptamer switch design for biorecognition interface 125 whereby the biomarker competitively binds to the ssDNA aptamer, resulting in the release of a molecule including a second ssDNA and the electroactive label. The surface of the working electrode 155 can be modified with capture ssDNA molecules configured to bind to these released molecules for detection. In other implementations, the aptamer can be a ribonucleic acid (RNA), a xeno nucleic acid (XNA), or a peptide. In a particular implementation, the electroactive label chemical is an electroactive redox label such as methylene blue (MB), thionine, or ferrocene.

FIG. 1C is a schematic diagram illustrating components of the assembled aptamer sensor patch 100, in accordance with some implementations of the disclosure. FIG. 1D is a schematic diagram showing a structure and valve operation of a microfluidic module 160 of the assembled aptamer sensor patch 100. The microfluidic module 160 includes various fluidically coupled components, including an inlet 161 for receiving a biofluid/sweat sample, a sensing reservoir 162 for capture and quantification of biomarkers contained in the biofluid sample, microvalves 163a-163b for routing the biofluid sample, and an outlet 164 that provides a channel for an outflow of the biofluid sample. In this example implementation, microvalves 163a-163b are configured to precisely control biofluid sampling. When induced sweat enters the microfluidic channel via inlet 161 and travels into sensing reservoir 162, biomarker analysis can initiate while newly secreted sweat is rerouted to outlet 164 due to the capillary effects of microvalves 163a-163b. By virtue of such a microfluidic valving design, highly stable biosensing can take place in the quiescent sweat matrix free from the influence of sweat flow.

Diagrams 172-176 of FIG. 1D schematically illustrate the operation of microvalves 163a-163b, in accordance with a particular implementation of the disclosure. The microvalves in this implementation are configured as CBVs that stay closed and block sweat at pressures lowers than their bursting pressures (BPs). In this geometric design, microvalve 163a has a lower BP than microvalve 163b. As shown by diagrams 172-174, sweat can first flow into the microfluidic module 160 via inlet 161 and enter the main sensing reservoir 162. At this point the sweat arrives at the at the two CBVs in their closed states. Once sweat fully fills the sensing reservoir 162, the pressure at the two microvalves increases and reaches the BP of microvalve 163a first. As illustrated by diagram 176, at this point, microvalve 163a can open to allow new sweat to flow through and eventually flow out through the outlet 164. During the entire sensing period, microvalve 163b can remain closed. Microvalve 163b can function to balance air pressure and allow sweat to enter and fill the sensing reservoir 162 first. In some implementations, microvalve 163b can have a smaller diameter than microvalve 163a.

Although the foregoing illustrates the microvalve design and operation in one particular implementation, it should be appreciated that other geometric designs could be implemented. For example, in some implementations a single microvalve could be used to reroute newly secreted sweat to outlet 164 when the sensing reservoir 162 is filled. In some implementations, other microvalve designs besides CBVs could be used to direct sweat flow.

In addition to components of microfluidic module 160 of the assembled aptamer sensor patch 100, FIG. 1C depicts sensing components of the aptamer sensor assembly. Such components can include a reference electrode (RE) 153 of sensor assembly 150, a counter electrode (CE) 154 of sensor assembly 150, and an aptamer sensor 158 comprised of working electrode 155 facing biorecognition interface 125. In some implementations, parallel faces of the working electrode 155 and biorecognition interface 125 can function as walls of the microfluidic sensing reservoir 162 that are bridged on sweat filling. It should be noted that although implementations illustrated herein show the surfaces of working electrode 155 and biorecognition interface 125 facing each other, in other implementations the aptamer sensor patch 100 can be manufactured such that the working electrode 155 and biorecognition interface 125 are positioned in other orientations relative to one another, including being situated adjacent to one another.

In some implementations, biosensor device 300 can be configured to apply an electric field between working electrode 155 and counter electrode 154 to shorten the incubation time for biomarker analysis by enhancing molecular transport of the electroactive label chemicals to the working electrode 155. For example, a positive potential bias could be applied between the working electrode 155 and counter electrode 154. In such implementations, the electroactive label chemical can be carried by a negatively charged molecule (e.g., ssDNA), such that applying a positive potential at the working electrode 155 can promote electrophoresis-based enhanced transport of the released redox probe (e.g., MB-ssDNA) across the sensing gap, substantially reducing the necessary incubation time.

Additional sensors that can be included in the sensor assembly 150 are a pH sensor 156, and an ionic strength sensor 157. In one implementation, pH sensor 156 is a potentiometric sweat pH sensor. In one implementation, ionic strength sensor is an impedimetric ionic strength sensor. Although not illustrated in this example, a further sensor that could be included as part of sensor assembly 150 is a skin temperature sensor. Alternatively, the skin temperature sensor can be incorporated in FPCB 200. Having additional, integrated pH, ionic strength, and/or temperature, sensors can enable real-time personalized biomarker data calibration to mitigate the interpersonal sample matrix variation-induced sensing error, and provide a more comprehensive assessment of the physiological status of the wearer of biosensor device 300. In some implementations, the combination of sensors of sensor assembly 150 can be implemented as a multiplexed sensor array. In other implementations, some of the additional sensors can be excluded, or other additional sensors can be included to enable calibration.

The foregoing design of aptamer sensor patch 100 can enable automatic, highly sensitive, and efficient electrochemical detection of trace-level sweat biomarkers such as proteins or steroid hormones, including estradiol, in situ on the skin. For example, in some implementations, the aptamer sensor assembly including sensory assembly 150 and biorecognition interface 125 can be configured to determine the concentration of the biomarkers with a sensitivity of 100 picomoles or less, 10 picomoles or less, or even less than 1 picomole. In some experiments, further described below, a sensitivity of 0.14 pM was observed.

In some implementations, the aptamer sensor patch 100 can be designed to eliminate leakage of a sweat sample. For example, the electrostimulation may be applied to several neighboring sweat glands while avoiding the sweat glands directly underneath an inlet. The patch can be designed to allow for collection of a sweat sample from only glands not in touch with the hydrogels and prevent leakage of sweat from the neighboring sweat glands (which mixed with hydrogel). This can be achieved through application of pressure on the gland the sample is taken from and through application of specialized adhesive taping of the neighboring glands and use of secure adhesive to attach the skin patch. The application of hydrogel can also be limited to optimal parts of the patch to minimize interference.

The FPCB 200 can be configured for iontophoretic sweat induction, sensor data acquisition, sensor data processing or other signal processing, and/or wireless communication with a mobile device 50. The FPCB could also incorporate a skin temperature sensor to provide skin temperature information during in situ sweat sensing. During assembly, the FPCB 200 can removably couple to the patch 100 to form the fully integrated wearable biosensor device 300. The FPCB 200 can be configured as a reusable electronic system that interfaces with disposable, point-of-care aptamer sensor patches 100. A battery 217 (e.g., lithium battery) can power the system, enabling functions such as signal processing and wireless communication. In other implementations, the biosensor device 300 can be powered by other or additional means such as by human motion, solar cells, and/or by a biofluid powering system that powers the device using collected sweat flow. As such, by utilizing efficient wearable energy harvesting systems such as biofuel cells and/or solar cells, the wearable biosensor device 300 could be realized as a fully self-powered wearable system.

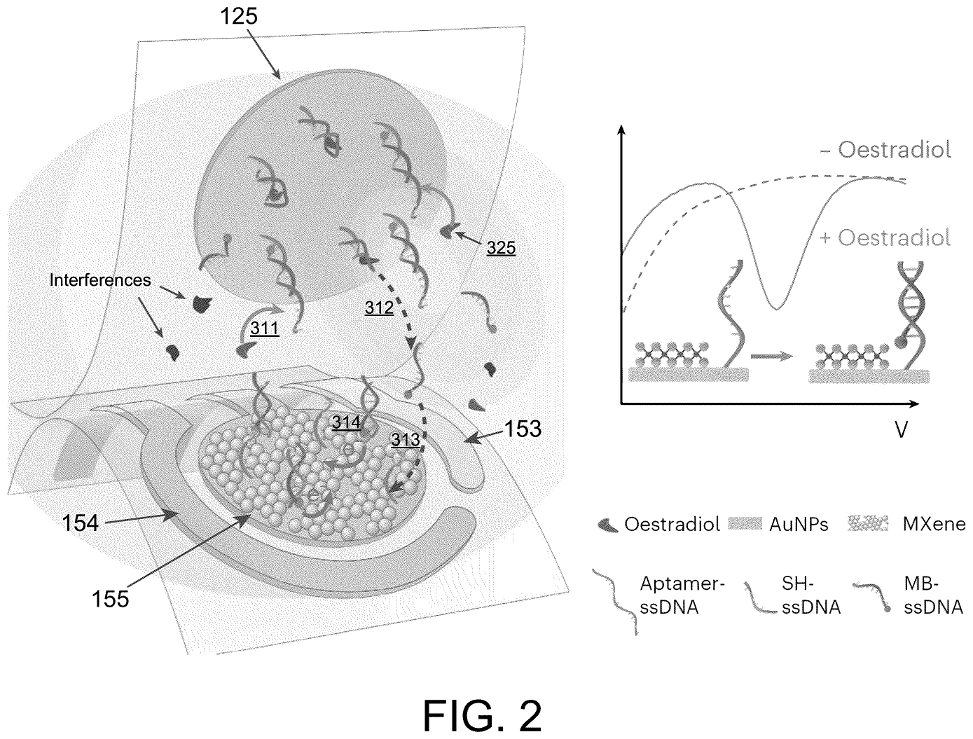

FIG. 2 is a schematic diagram conceptually illustrating a particular embodiment of the operation of an aptamer sensor assembly that is configured to provide the reagentless in situ quantification of the biomarker estradiol, in accordance with the disclosure. In this embodiment, a surface of biorecognition interface 125 faces a surface of working electrode 155. The working electrode 155 is configured as a AuNPs-MXene sensor, and the biorecognition interface 125 is configured as a target-induced strand displacement aptamer switch. Step 311 represents recognition of the estradiol molecule 325 by the aptamer on the biorecognition interface 125. Step 312 represents target recognition induced strand displacement to release the MB-ssDNA. Step 313 represents recapture of the released MB-ssDNA by capture thiolated ssDNA (SH-ssDNA) on the working electrode 155. Step 314 represents electrochemical quantification of the methylene blue from the recaptured MB-ssDNA on the working electrode 155. Also illustrated are reference electrode 153 and counter electrode 154.

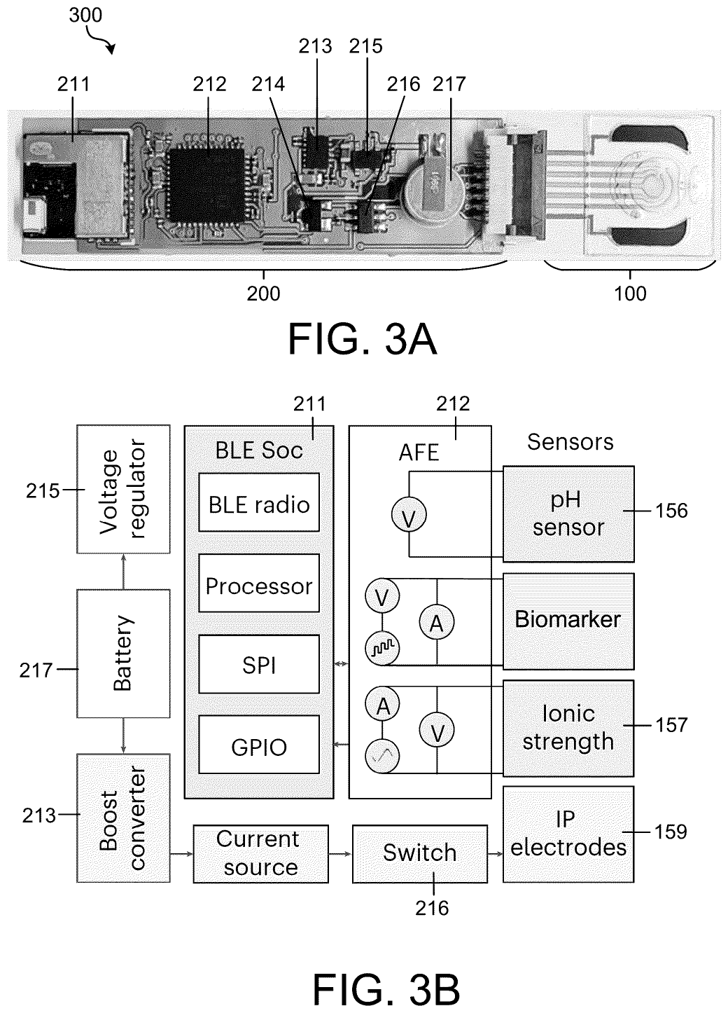

FIG. 3A shows a photograph of a flatted electronic system of a particular embodiment of a biosensor device 300, in accordance with some implementations of the disclosure. FIG. 3B shows a corresponding block diagram of the electronic system of the embodiment of FIG. 3A. As depicted by FIG. 3A, the circuit components of FPCB 200 can include a wireless communication module 211, an electrochemical analog front-end (AFE) 212, a boost converter 213, a bipolar junction transistor array 214, a voltage regulator 215, an analog switch 216, and/or a battery 217. The wireless communication module 211 can integrate a microcontroller for signal processing and wireless communication. As illustrated by FIG. 3B, the wireless communication module 211 can be implemented as a programmable BLE system-on-chip (Soc) having a BLE radio, processor, serial peripheral interface (SPI), and general-purpose input and output (GPIO). The electrochemical AFE 212 can enable multiplexed sensing by performing multimodal electrochemical measurements such as voltammetry for biomarker (e.g., estradiol) sensing, impedimetry for pH sensing, and/or potentiometry for ionic strength sensing. The electrochemical AFE 212 can also provide skin temperature information continuously via a built-in temperature sensor. During in situ sweat analysis, the obtained pH, ionic strength and/or skin temperature levels can be used to calibrate the biomarker sensor readings in real-time. The boost converter 213, bipolar junction transistor array 214, and analog switch 216 can provide iontophoretic sweat induction. The voltage regulator 215 can provide power management. The battery 217 can power the circuit components. Via the illustrated electronics of this embodiment, a FPCB can be configured to perform current-controlled iontophoresis, multiplexed electrochemical measurements (including voltammetry, impedimetry, and/or potentiometry), signal processing, and wireless communication. The system could also accurately obtain the dynamic responses of pH, ionic strength, and skin temperature sensors for real-time biomarker sensor calibration.

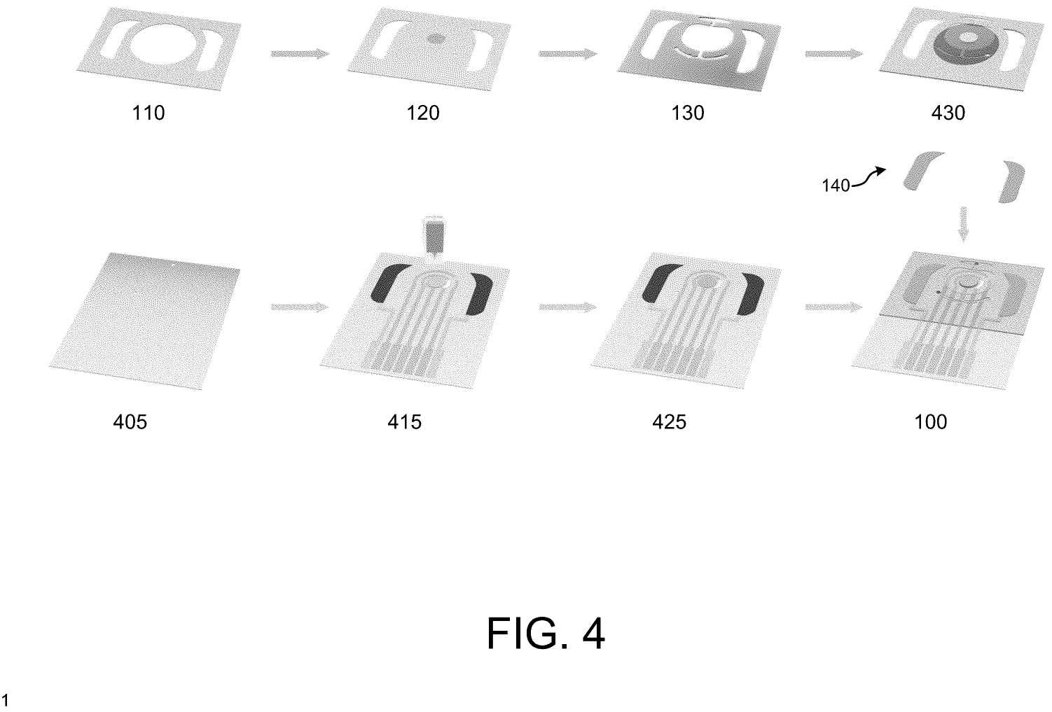

FIG. 4 is a schematic diagram illustrating an example method of assembling a aptamer sensor patch 100, in accordance with some implementations of the disclosure. A microfluidics assembly that is flexible can be assembled by stacking laser-cut layers 110-130. Cutouts can be formed in layers 110-130 for a sweat accumulation reservoir, one or more inlets, a sensing/detection reservoir, one or more outlets, one or more channels, microvalves, hydrogel, and/or other components of the aptamer sensor patch 100. In this particular example, a bottom accumulation layer 110 can be patterned with a sweat accumulation reservoir and microchannels including CBVs. The biorecognition interface layer 120 can be patterned with an inlet, and it can have a biorecognition interface surface formed thereon via inkjet-printing of nanoparticles (e.g., AuNPs). Thereafter, the surface of the created biorecognition interface can be modified for biomarker detection. In some implementations, the surface can be modified to detect one type of biomarker. In other implementations, the surface can be modified to detect multiple different types of biomarkers (e.g. two different female hormones). A collection layer 130 can be cut to make an inlet and outlet, channels with CBVs, and a sweat collection reservoir.

The accumulation layer 110 can be a patterned medical adhesive such as medical tape that can be double-sided. The biorecognition interface layer 120 can be formed of a thermoplastic polymer resin such as Polyethylene terephthalate (PET), with the nanoparticles thereon. The collection layer 130 can include an assembly of a thermoplastic polymer resin sandwiched between double-sided medical adhesive such as medical tape. As depicted, the biorecognition interface layer 120 can be stacked/adhered over the accumulation layer 110, and the collection layer 230 can be stacked/adhered over biorecognition interface layer 120 to form microfluidics assembly 430.

Also depicted in FIG. 4 is a substrate layer 405 (e.g., PET layer) on which electrodes of sensor assembly 150 can be printed or otherwise created to form assembly 415. Thereafter, the surface of the created working electrode can be modified for biomarker detection. In some implementations, the surface can be modified to enable detection of one type of biomarker. In other implementations, the surface can be modified to enable detection multiple different types of biomarkers. During further assembly of aptamer sensor patch 100, assemblies 425 and 430 can be stacked/adhered such that the biorecognition interface and working electrode are face-to-face, and the hydrogel agent 140 can be applied.

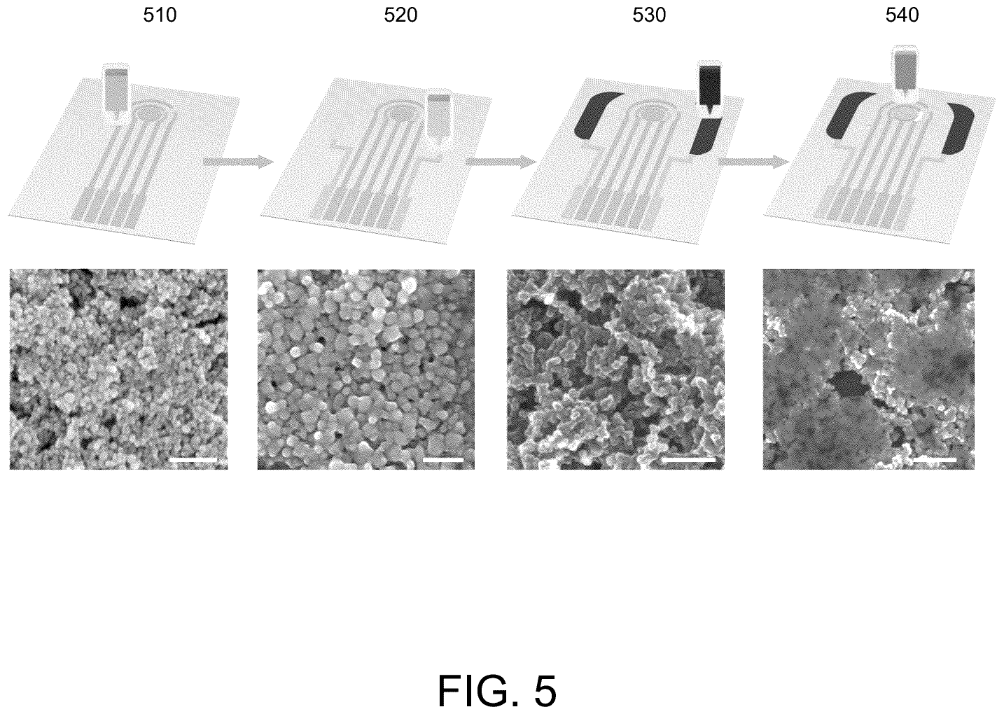

FIG. 5 is a schematic diagram illustrating an example method of inkjet printing components of a sensor assembly 150 on a substrate layer 405 to form assembly 415, and corresponding scanning electron microscope characterizations of printed nanostructure patterns at different stages of inkjet printing, in accordance with a particular embodiment of the technology described herein. In the optical images, the scale bar is 200 nm. At stage 510, gold is printed in one or more layers to form reference, working, and counter electrodes, a pH sensor, and an ionic sensor. At stage 520, silver is printed to form interconnects and one or more additional layers of the reference electrode. At stage 530, carbon is printed to form iontophoresis electrodes. At stage 540, MXene is printed for the working electrode in one or more layers over the one or more gold layers formed at stage 510 for the working electrode. In other implementations, other suitable metals or materials can be printed to form the different layers of the components of sensor assembly 150. By virtue of this printing process, the disposable sensor patch can be mass produced with scalable manufacturing. Printable wearable sensor patches can be fabricated on a large scale at a relatively low cost. This can allow for disposable sensor patches that can be worn by an individual for an extended of time (e.g., 12-24 hours), which can be replaced on a daily level, and which can collect health information without invasive testing and the need for a human patient to come into a physical laboratory for repeated testing.

It should be appreciated that other methods of assembly are contemplated other than the ones described and illustrated with reference to FIGS. 4-5, and that other biosensor assemblies besides wearable patches are contemplated as being in accordance with the technology described herein. Components of the biosensor device 300, including one or more of the components of the FPCB 200 and aptamer sensor patch 100 could instead be integrated into a wearable device such as a smartwatch, a ring, or a HMD. For example, components of the FPCB 200 and aptamer sensor patch 100 could be incorporated into an area of a smartwatch or ring that contacts a user's skin. A smartwatch incorporating components of aptamer sensor patch 100 could itself run an application that displays health information associated with the collected data, and/or alternatively communicate the data to another mobile device 50 such as a smartphone or wearable HMD that runs an application as described above.

Although the foregoing examples are described in the context of a biosensor device 300 that incorporates an iontophoresis module that induces sweat in the user, it should be appreciated that a biosensor device 300 can be designed without an iontophoresis module and its associated components (e.g., without iontophoresis electrodes and/or carbagel). In such implementations, a sweat sample could be induced and collected by exposing the user to heat, by the user exercising, or by some other stimuli including, but not limited to, the environment, the user's actions, and/or an external device.

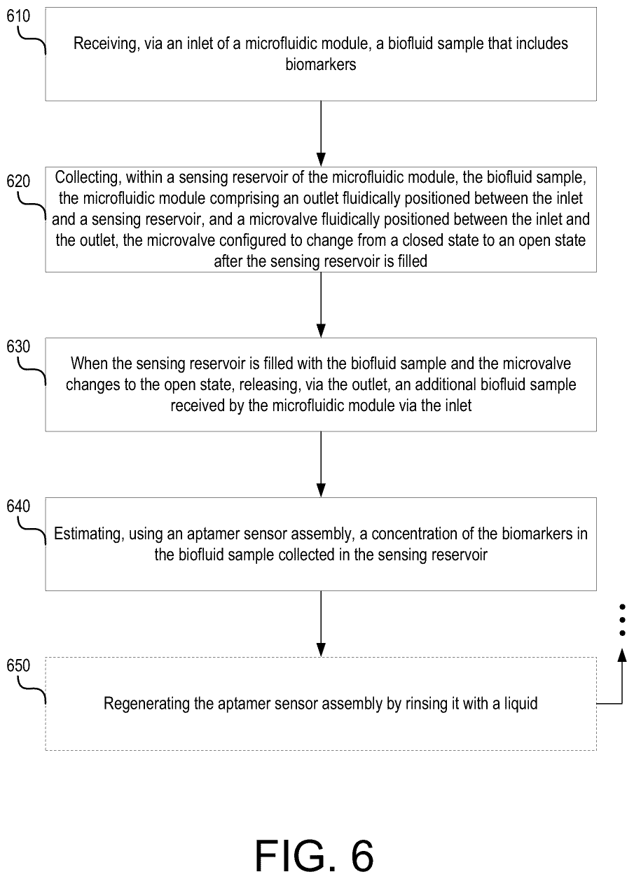

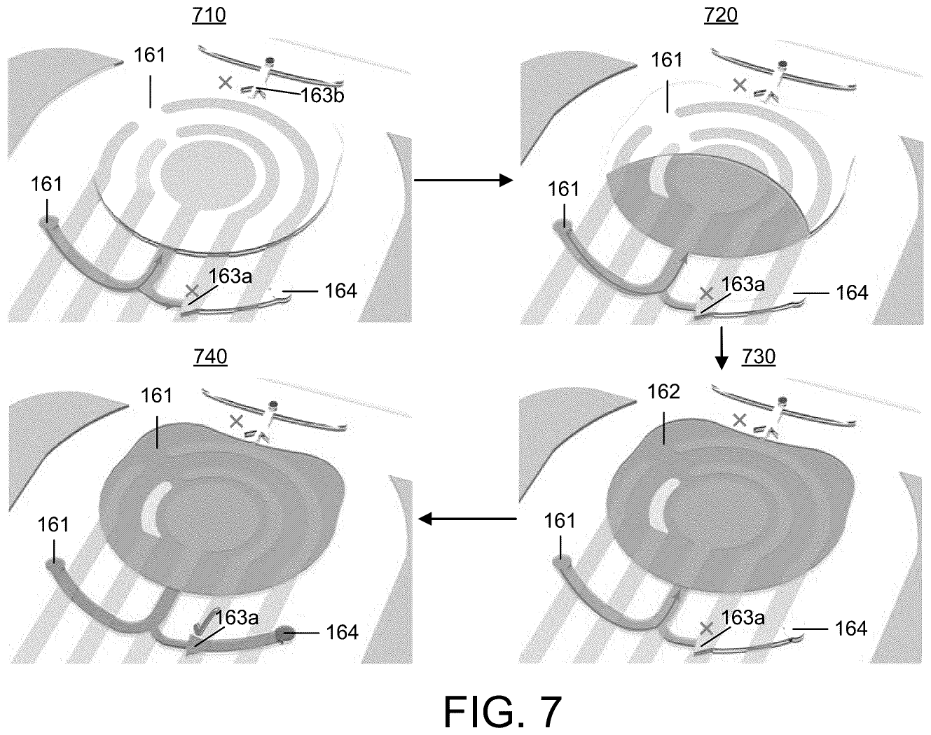

FIG. 6 is an operational flow diagram example operations performed in a microfluidic module of aptamer sensor device during automatic and controlled biofluid sampling, in accordance with some implementations of the disclosure. For example, operations of FIG. 6 can be performed using components of aptamer sensor patch 100 illustrated in FIGS. 1C-1D. FIG. 6 will be described with reference to FIG. 7 which is a schematic diagram illustrating different stages of biofluid sampling in a particular microfluidic valving design, corresponding to FIG. 1D, for realizing a precise sampling of a biofluid sample for biomarker detection. However, it should be appreciated that the technology described herein could be implemented using other microfluidic valving designs.

Operation 610 includes receiving, via an inlet of a microfluidic module, a biofluid sample that includes biomarkers. For example, as illustrated by stage 710 (sweat entry) of FIG. 7A, an inlet 161 can receive the biofluid sample. The biofluid sample can be a sweat sample. In some implementations, the sweat sample can be autonomously induced using an iontophoresis module as described above (e.g., using electrodes 159 and hydrogel agent 140). The sweat sample can be induced by other means, including exercise and/or the environment. In some implementations, the biomarkers are reproductive hormones such as estradiol or progesterone.

Operation 620 includes collecting, within a sensing reservoir of the microfluidic module, the biofluid sample. The microfluidic module comprises an outlet fluidically positioned between the inlet and the sensing reservoir, and a microvalve fluidically positioned between the inlet and the outlet, the valve configured to change from a closed state to an open state after the sensing reservoir is filled. For example, as illustrated by FIG. 7, sensing reservoir 162 is configured to be filled with the biofluid sample received via inlet 161. Fluidically positioned between inlet 161 and sensing reservoir 162 is an outlet 164. As illustrated by stage 720 (sweat collection) and stage 730, a microvalve 163a fluidically positioned between inlet 161 and outlet 164 is configured to be in a closed state while the sensing reservoir 162 is being filled with the biofluid sample.

Operation 630 includes when the sensing reservoir is filled with the biofluid sample and the valve changes to the open state, releasing, via the outlet, an additional biofluid sample received by the microfluidic module via the inlet. For example, as illustrated by stage 740 of FIG. 7, once sensing reservoir 162 is filled with the biofluid sample, microvalve 163a can open and any new biofluid sample received via inlet 161 can be rerouted via outlet 164. To perform this operation, microvalve 163a can be configured as a CBV having a BP that is exceeded when the sensing reservoir 162 is filled. In this illustrated example, the microfluidic module has two microvalves that can act as CBV-microvalve 163a and 163b. Microvalve 163b can remain closed throughout the process, and function to balance air pressure in the microfluidic module.

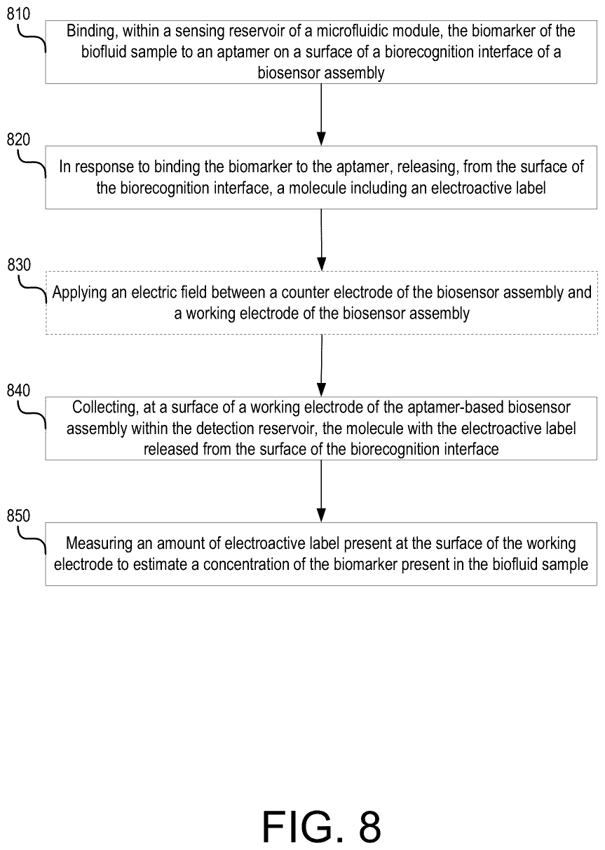

Operation 640 includes estimating, using an aptamer sensor assembly, a concentration of the biomarkers in the biofluid sample collected in the sensing reservoir 162. To this end, the aptamer-based biosensor assembly can include a biorecognition interface including aptamers that bind to the biomarkers, and a working electrode that measure electroactive labels released from the biorecognition interface when the aptamers bind to the biomarkers. Particular techniques for performing biosensing using an aptamer assembly are further described below with reference to FIG. 8. By virtue of rerouting any new biofluid received via the inlet to the outlet while biosensing is performed, highly stable biosensing can take place without the influence of biofluid flow.

Optional operation 650 includes regenerating the aptamer sensor assembly by rinsing it with a liquid. The microfluidic module can be flushed with a deionized water or a solution with low ionic strength or acidic pH. The microfluidic module can be flushed for about 3 minutes or less, 2 minutes or less, 1 minute or less, or 30 seconds or less. By virtue of regeneration, repetitive biomarker quantification could be performed using the same aptamer sensor assembly. Regeneration can be particularly suitable in embodiments where sensing is realized using a biomarker-induced strand displacement reaction that releases molecules containing electroactive labels. In such implementations, electrostatic adsorption can only contribute positively to sensitivity without compromising sensor selectivity.

In some implementations, continuous wearable biomarker sensing could be realized through integrating a multi-reservoir CBVs design biomarker sensor arrays into a single sensor patch. In some implementations, sweat samples can be collected without reapplication of a hydrogel agent for a period of time. In some implementations, sweat samples can be periodically or continuously collected in the microfluidic module, channeled into a sensing reservoir, analyzed, and then flushed out through an outlet. After a full day or other time period, a new aptamer sensor patch with a new hydrogel agent may be applied and the foregoing process for biomarker detection repeated. The process can be repeated on a daily basis for an extended period of several days, weeks, or even months. The process can also be resumed after a break of a period of days, weeks, or months, to evaluate a change in hormone levels or some other health characteristic of the user.

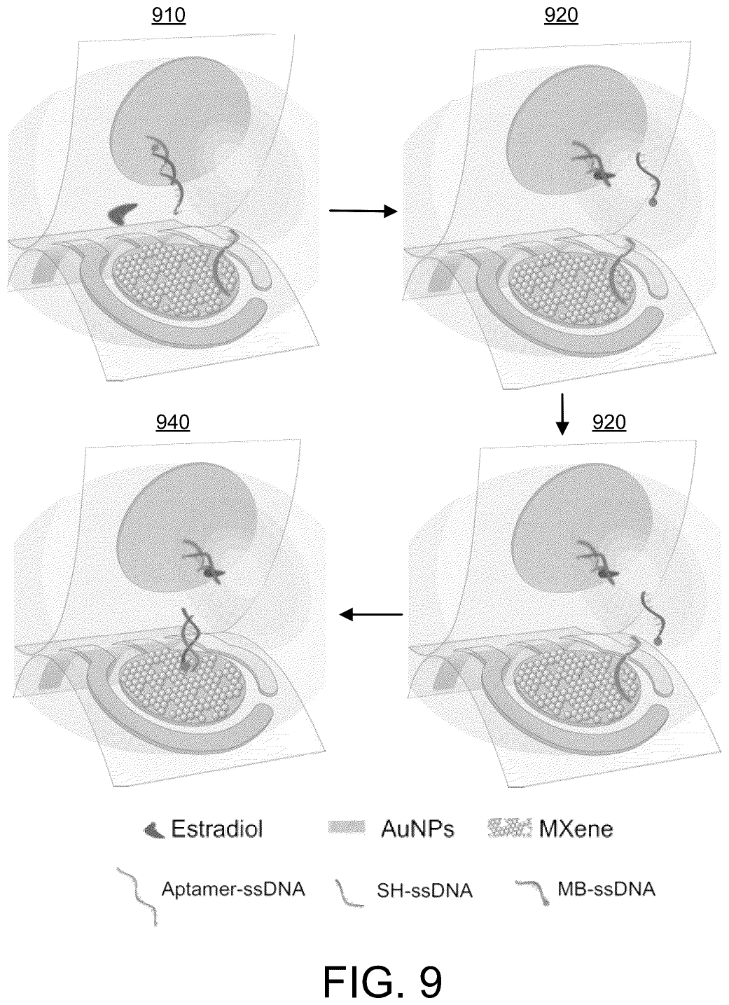

FIG. 8 is an operational flow diagram illustrating example operations performed by an aptamer sensor assembly of a biosensor device during reagentless, automatic, and in-situ recognition and analysis of biomarkers contained in a biofluid sample, in accordance with some implementations of the disclosure. The aptamer sensor assembly includes a biorecognition interface including aptamers that bind to the biomarkers, and a working electrode that measures electroactive labels released from the biorecognition interface when the aptamers bind to the biomarkers. FIG. 8 will be describe with reference to FIG. 9, which is a schematic diagram illustrating the operation of an aptamer sensor assembly that is configured to provide the reagentless in situ quantification of the biomarker estradiol using a biorecognition interface modified with an estradiol-selective DNA aptamer facing a AuNPs-MXene-based detection working electrode modified with capture ssDNA. However, it should be appreciated that the biosensor devices described herein can be configured to realize automatic detection in sweat of other biomarkers besides estradiol, especially biomarkers that could be present in low (e.g., picomolar or subpicomolar) concentrations, including other steroid hormones, proteins, and the like. Moreover, it should be appreciated that aptamers other than ssDNA could be utilized with the technology described herein.

Operation 810 includes binding, within a sensing reservoir of a microfluidic module, a biomarker of the biofluid sample to an aptamer on a surface of a biorecognition interface of the biosensor assembly. Prior to performing the method of FIG. 8, the surface of the biorecognition interface can be modified with aptamer that selectively binds to the biomarker. For example, the surface can be modified with a female hormone selective aptamer that binds to estradiol or progesterone. In a particular implementation, the aptamer is ssDNA. For example, stage 910 of FIG. 9 illustrates recognition of an estradiol molecule by an ssDNA aptamer on a AuNPs biorecognition interface.

Operation 820 includes in response to binding the biomarker of the biofluid sample to the aptamer, releasing, from the surface of the biorecognition interface, a molecule with an electroactive label. In addition to modifying the surface of the biorecognition interface with an aptamer that selectively binds to the biomarker, the surface can be modified with a molecule containing an electroactive label chemical (e.g., MB) that enables electrochemical quantification. The biomarker-selective aptamer can be partially hybridized with this molecule. In the presence of the biomarker, the molecule with the electroactive label can be released because of the higher affinity of the aptamer to the biomarker than the partially hybridized sequence. For example, stage 920 of FIG. 9 illustrates target recognition-induced strand displacement at the biorecognition interface to release MB-ssDNA partially hybridized to the aptamer.

Optional operation 830 includes applying an electric field between a counter electrode of the biosensor assembly and a working electrode of the biosensor assembly. This can shorten the incubation time for biomarker analysis by enhancing molecular transport of released molecules containing the electroactive label to the working electrode. A positive potential bias could be applied between the working electrode to promote electrophoresis-based enhanced transport of the released molecule across the sensing gap, substantially reducing the incubation time needed to generate an adequate sensor signal. In some implementations, the electric field can be applied before incubation as a bias potential for a duration of 2 minutes or less, 1 minute or less, or 30 seconds or less.

Operation 840 includes collecting, at a surface of a working electrode of the aptamer-based biosensor assembly within the detection reservoir, the molecule with the electroactive label released from the surface of the biorecognition interface. Prior to performing the method of FIG. 8, the surface of the working electrode can be modified with a capture molecule that binds to the molecule containing the electroactive label. For example, the capture molecule can contain a ssDNA sequence that anneals via complementary DNA hybridization to the ssDNA sequence of the molecule containing the electroactive label. For example, stage 930 of FIG. 9 illustrates recapture of the released MB-ssDNA by SH-ssDNA on a surface of the working electrode.

Operation 850 includes measuring an amount of electroactive label present at the surface of the working electrode to estimate a concentration of the biomarker in the biofluid sample. For example, stage 940 of FIG. 9 illustrates electrochemical quantification of the MB from the recaptured MB-ssDNA on the working electrode. In this embodiment, it was observed that an AuNPs-MXene printed surface enhanced signal transduction (Signal ON) by enhancing the working electrode's electroactive surface area and charge transfer efficiency.

Any one of a number of voltammetric techniques that correlate current to concentration can be applied to make the measurement of the amount of electroactive label bound at the electrode surface. For example, SWV, DPV, linear sweep voltammetry (LSV), or some other voltammetric technique can be used to make the measurement. In a particular implementation, the recaptured molecules can be quantified by the redox signal measured electrochemically via a SWV. The reagentless “signal-on” detection approach, coupled with highly sensitive low-background SWV measurements, can off very high sensitivity (e.g., picomolar or subpicomolar) and applicability for ultra-low-level sweat biomarker analysis in situ.

Depending on the binding environment, there may be significant interpersonal and/or intrapersonal variations in the composition of the biofluid sample, which could affect the rate that biomarkers bind to the biorecognition interface and affect the accuracy of the estimated concentration of the biomarker. For example, it was observed that pH, electrolyte concentration, and skin temperature can influence the sensor readout of estradiol concentration expressed as a current measurement. As such, in some implementations, to further improve the quantification of biomarkers contained in the biofluid sample, the influence of temperature, pH, and/or ionic strength on the biomarker sensor readings can be calibrated in real-time based on readings from a temperature sensor, pH sensor, and/or ionic strength sensor of the biofluid sample in the sensing reservoir.

Although some of the foregoing examples have been described in the context of a wearable biosensor device that uses a reagentless aptamer sensor assembly to sense one type of biomarker present in sweat or some other biofluid sample, the biosensor devices described herein can be adapted to sense multiple different types of biomarkers present in a sweat sample. To that end, in some implementations the surface of the biorecognition interface and/or the surface of the working electrode can be functionalized to sense multiple different types of biomarkers. For example, in one implementation the surfaces can be functionalized to sense at least two different types of female hormones. For example, both estradiol and progesterone could be sensed and quantified from a sweat sample.

In some implementations, sensing of at least two different types of biomarkers present in a sweat sample can be achieved by an aptamer sensor assembly having a biorecognition interface with a surface functionalized with a first aptamer that binds to a first type of biomarker, and a second aptamer that binds to a second type of aptamer. The biorecognition interface surface can be further functionalized with a first labeled molecule containing a first electroactive label, and a second labeled molecule containing a second electroactive label. In response to the first aptamer binding to the first type of biomarker, the biorecognition interface can be configured to release the first labeled molecule. In response to the second aptamer binding to the second type of biomarker, the biorecognition interface can be configured to release the second labeled molecule. The surface of the working electrode can be functionalized to bind to the first labeled molecule and the second labeled molecule. The first electroactive label and the second electroactive label can be different to enable sensor measurements of both the first and second types of biomarkers.

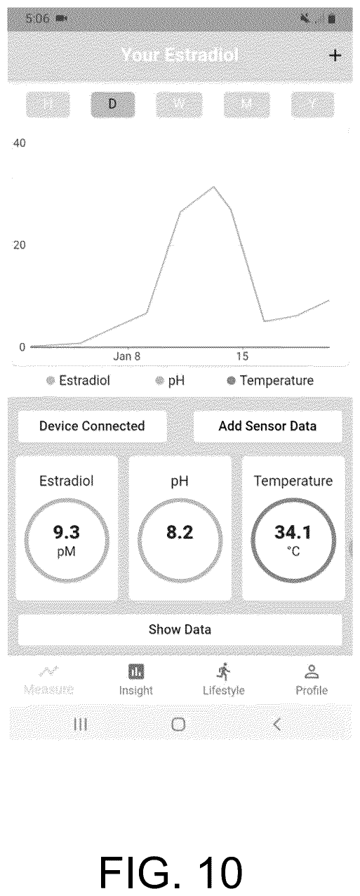

FIG. 10 illustrates an example GUI that can be presented to a user (e.g., patient) by running a mobile application used in conjunction with a wearable biosensor device 300 for noninvasive automatic biomarker monitoring, in accordance with some implementations of the disclosure. For example, the application can be run by a mobile device 50 wirelessly coupled to a biosensor device 300. During runtime, the GUI can display real-time data (processed or otherwise) acquired by the biosensor device 300. The GUI can also display historical data that was acquired. For example, based on a sweat sample collected by the biosensor device 300, data such as a estradiol concentration (e.g., in pM), a pH, and a skin temperature can be acquired and presented in real-time. The data can be plotted over time to provide an indication of the user's reproductive hormone levels or other biological levels over time. The GUI can provide an indication of whether the user's measured health data is within target range (e.g., via textual or visual markers). The GUI can also provide an indication of the status of the biosensor device 300 (e.g., whether it is presently connected to the mobile device).

In some implementations, the mobile application can itself perform, prior to user display, processing of sensor measurements received from a biosensor device 300. For example, in one implementation, the mobile application can be configured to convert a biomarker concentration based on an obtained voltammogram (e.g., SWV voltammogram) and corresponding real-time obtained values of calibration sensors such as an ionic strength sensor, pH sensor, and temperature sensor.

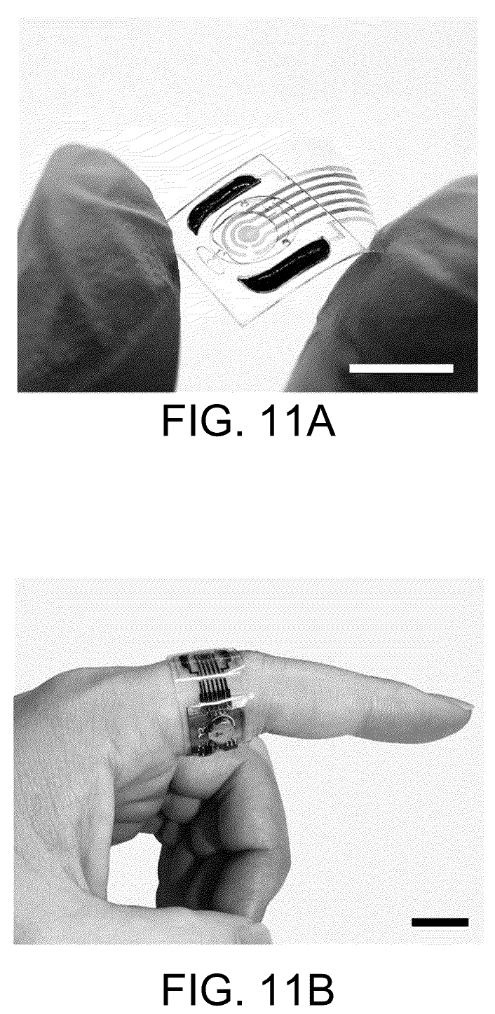

FIG. 11A shows a photograph of a particular embodiment of sensor patch 100 that is disposable. FIG. 11B shows a photograph of a particular embodiment of a biosensor device 300 that is implemented as a fully integrated wireless wearable patch worn on a finger for hormone monitoring. In the photographs of FIGS. 11A-11B, the scale bars are 1 cm.

### Experimental and Simulation Results

Various experiments and simulations were performed using a biosensor device 300 and/or components thereof used to wirelessly, autonomously, and non-invasively monitor estradiol levels, in accordance with a particular embodiment of the disclosure. The design of this particular biosensor device 300, and its associated experimental and simulations results, are further detailed below. Although these experimental and simulation results exemplify some of the advantages of utilizing the technology described herein, it should be appreciated that the disclosure is not limited by the discussion that follows, which describes results and observations of utilizing particular example embodiments. For example, besides estradiol, this wearable approach could be adapted to assess other trace-level reproductive hormone biomarkers on-demand. Additionally, the operation principle described herein could be readily adapted to survey a broad array of biomarkers (e.g., proteins, steroid hormones, etc.), including biomarkers that indicate some physiological status of the user.

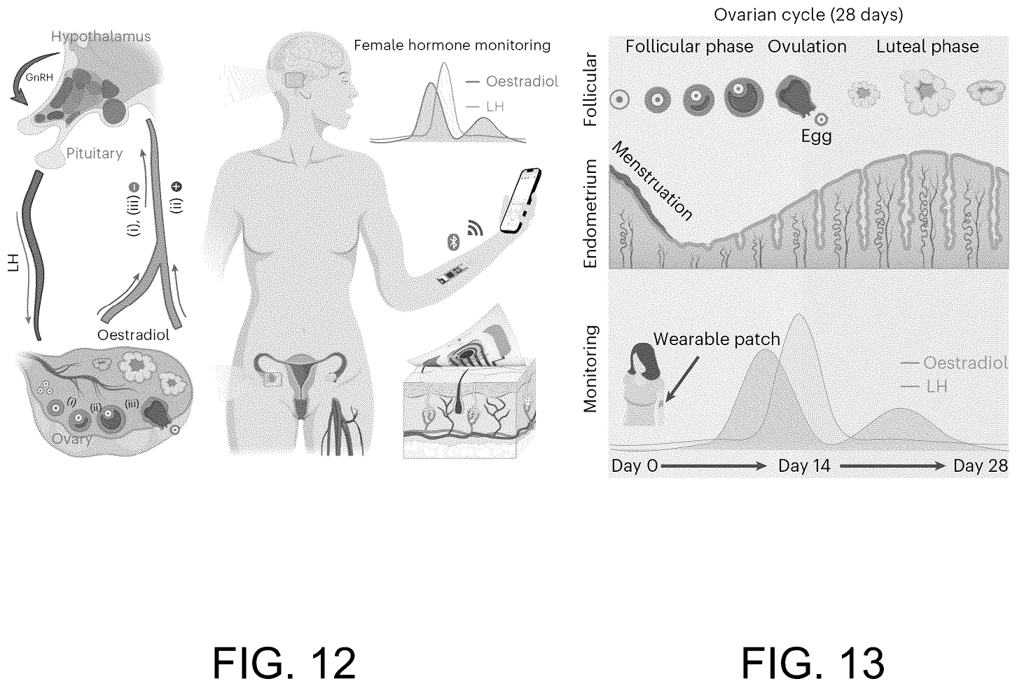

To appreciate the advantages of designing a reagentless, wearable, and estradiol biosensor device in accordance with particular embodiments, it is instructive to consider the importance of estradiol monitoring with reference to FIG. 12, which illustrates non-invasive monitoring of estradiol realized through sweat analysis using a skin-interfaced wearable sensor, in accordance with some embodiments. Female hormones affect much of women's health, from menstruation to pregnancy to menopause and more (for example, depression). They are regulated by the feedback mechanism of the hypothalamic-pituitary-ovarian axis, which governs all the female hormonal events related to reproductive activity. Estradiol is the primary form of the female hormone estrogen, and the most potent and prevalent female hormone during the reproductive years. As depicted by FIG. 12, which shows the (i) follicular phase, (ii) ovulation, and (iii) the luteal phase, during the menstrual cycle estradiol provides feedback to the hypothalamic-pituitary-ovarian axis to regulate the production of LH and ovulation. In addition to its centrality in sexual development, it markedly affects major organs, including the blood vessels, bones, muscles and brain, in both males and females. Therefore, estradiol monitoring is important in human biology from the cradle to the grave, and is an essential component of infertility management as well as general physiological surveillance. Compared to other alternative approaches for fertility monitoring based on the basal thermometer or urine luteinizing hormone, serum estradiol provides the most timely and accurate information. Moreover, serum estradiol analysis is a gold standard for monitoring the induction of ovulation with gonadotrophins; routine estradiol monitoring plays a crucial role for women in menopause and individuals undergoing hormone replacement therapy. However, despite the widespread importance of female hormones, the ability to measure them properly using conventional methods has not kept pace with their growing importance in clinical medicine. As such, there is a need for a wearable sensor based on a target-induced strand-displacement aptamer switch for in situ automatic electrochemical monitoring of female hormones with subpicomolar sensitivity as described in particular embodiments.

### In Vivo Evaluation of a Wearable Estradiol Sensor

A particular embodiment of a wearable estradiol sensor, further described below, was evaluated in vivo. During a menstrual cycle, estradiol level in the blood rises and falls twice as illustrated in FIG. 13. It gradually increases in the mid-follicular phase and reaches the highest point right before ovulation; then the estradiol level drops quickly after ovulation, followed by a secondary rise during the mid-luteal phase and a secondary decrease at the end of the menstrual cycle. Compared to invasive and lengthy blood assay, analyzing sweat estradiol using wearable technology offers a highly attractive approach for remote at-home female hormone monitoring.

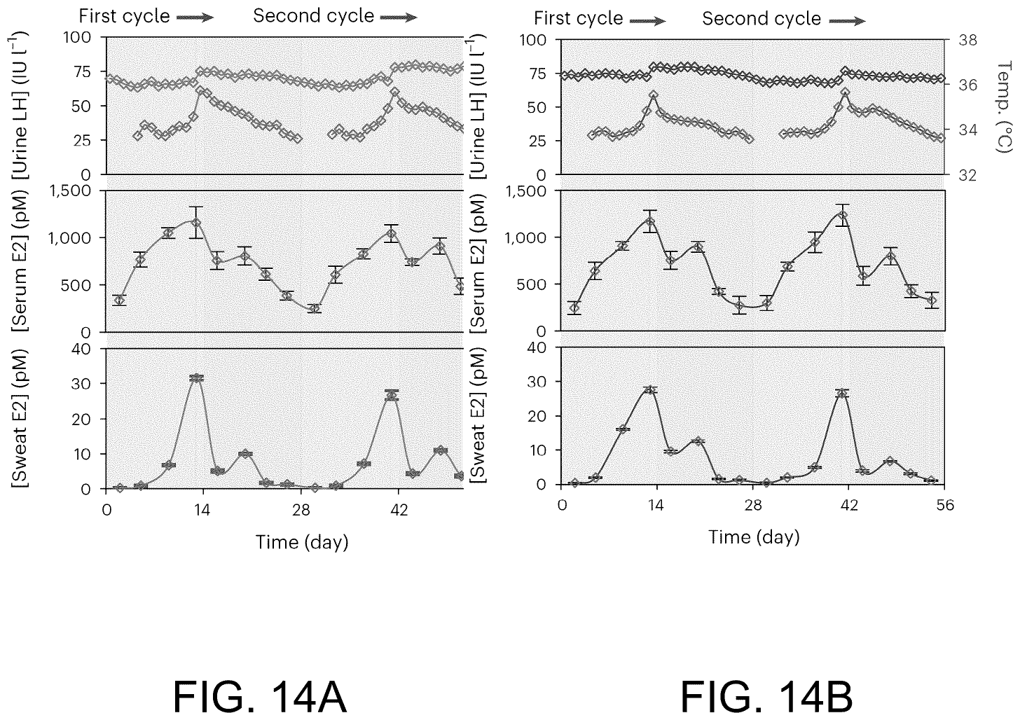

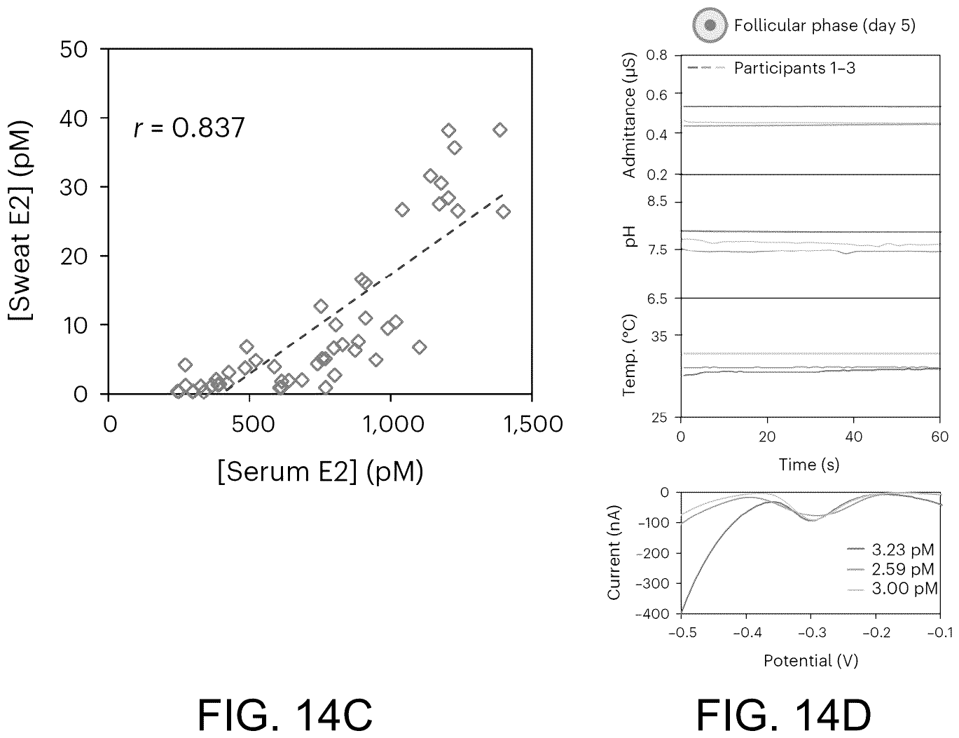

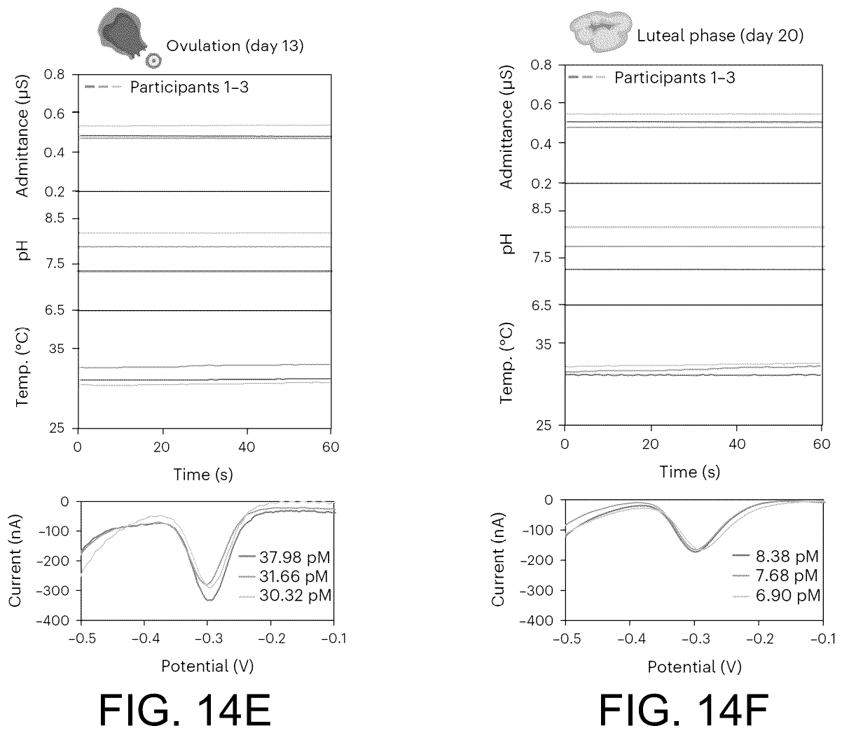

To validate the clinical values of the sweat estradiol measured by the sensor, human studies were conducted over two consecutive menstrual cycles on two healthy female participants (aged 28 and 32) by simultaneously monitoring urine luteinizing hormone, body temperature and blood estradiol. The results are depicted by the plots of FIGS. 14A-14B, where error bars represent the standard deviation of the mean from three measurements. The results revealed that estradiol levels in both sweat and serum reached the peak right before the ovulation while the urine luteinizing hormone and temperature levels peaked during the ovulation period, confirming estradiol's potential for early ovulation prediction. The main and secondary rise of the estradiol was also observed in both sweat and serum in all menstrual cycles. This indicated that sweat estradiol follows cyclical fluctuation during the menstrual cycles. In addition, a strong correlation coefficient of 0.837 was identified between sweat and blood estradiol levels from the pilot study (FIG. 14C), suggesting the high potential of sweat estradiol as a non-invasive biomarker for fertility and ovulation monitoring.

On-body evaluation of the wearable technology for real-time in situ estradiol analysis was performed on three female participants (aged 28, 31, and 32) on day 5, day 13 and day 20 during a menstrual cycle with the sensor patch conformally attached onto the skin. FIGS. 14D-F show plots corresponding to the results of the study. During the study, sweat was induced via the built-in iontophoresis module, and sampled by the microfluidics. Multiplexed sensor data were collected wirelessly using a user interface. The calibrated estradiol levels were converted in real time and displayed in a custom-developed mobile application (e.g., as depicted by FIG. 10) based on the obtained multimodal data. As expected, the lowest estradiol levels were observed on day 5 whereas the highest values appeared on day 13 in all participants; moderate estradiol levels were observed on day 20 due to the secondary rise of the estradiol during the menstrual cycle. Additional control studies were performed on three male participants; very low estradiol levels were obtained on all participants with no apparent fluctuations.

### Design and Characterization of Estradiol Nanobiosensor

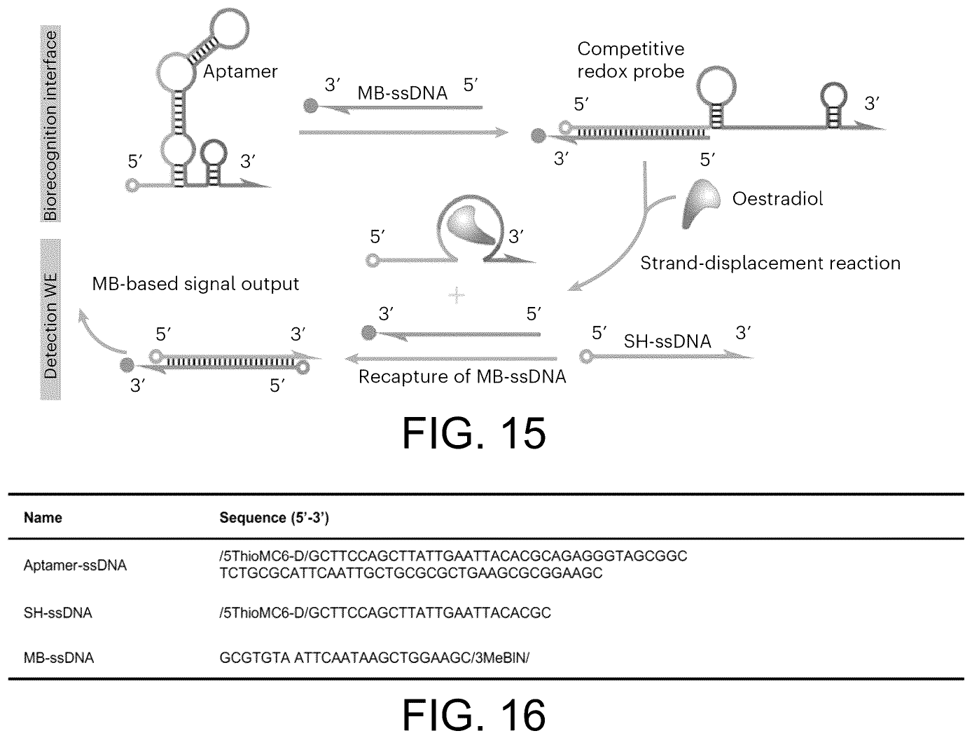

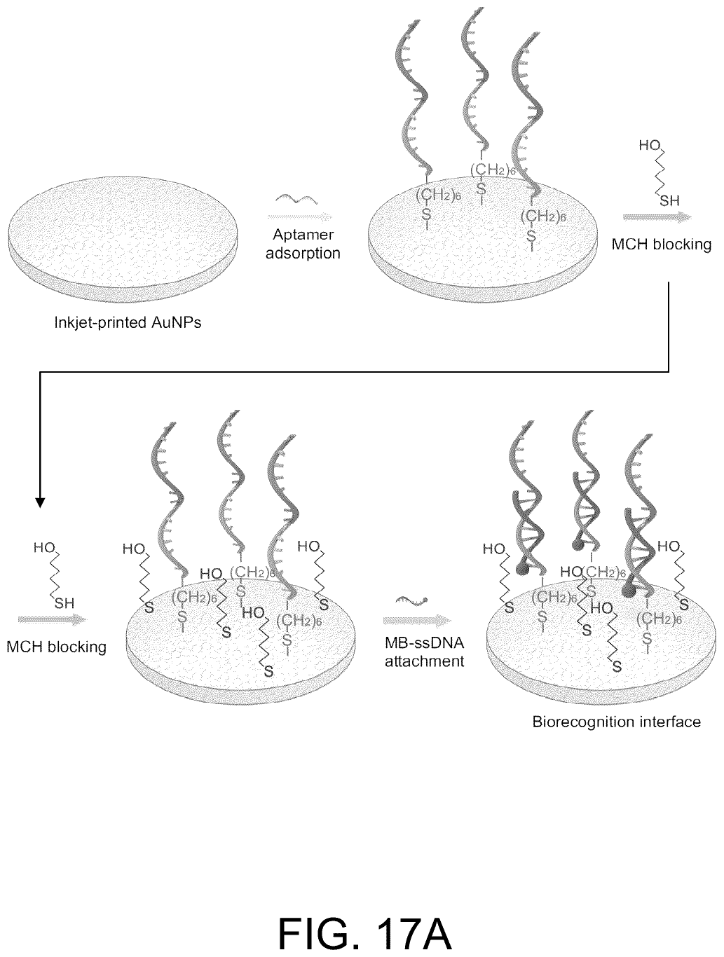

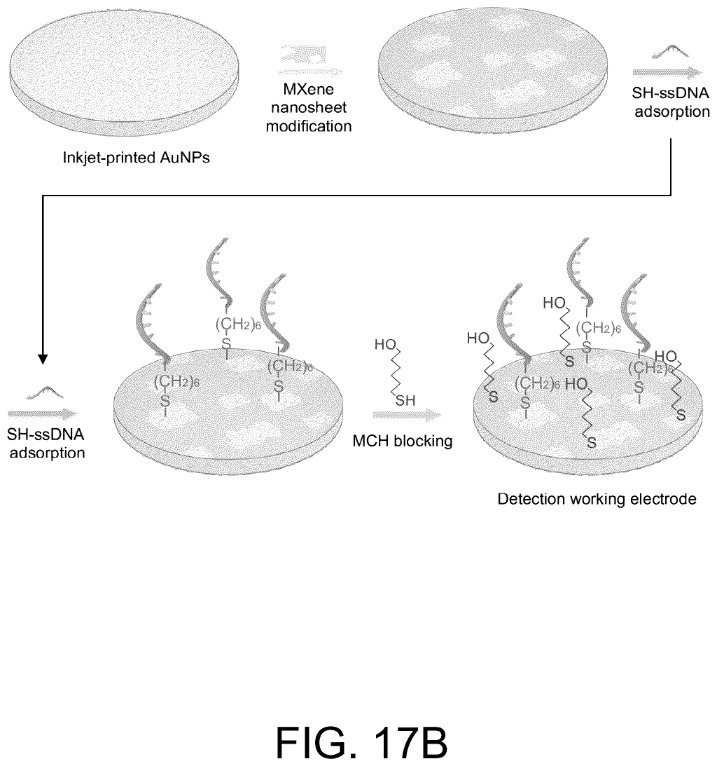

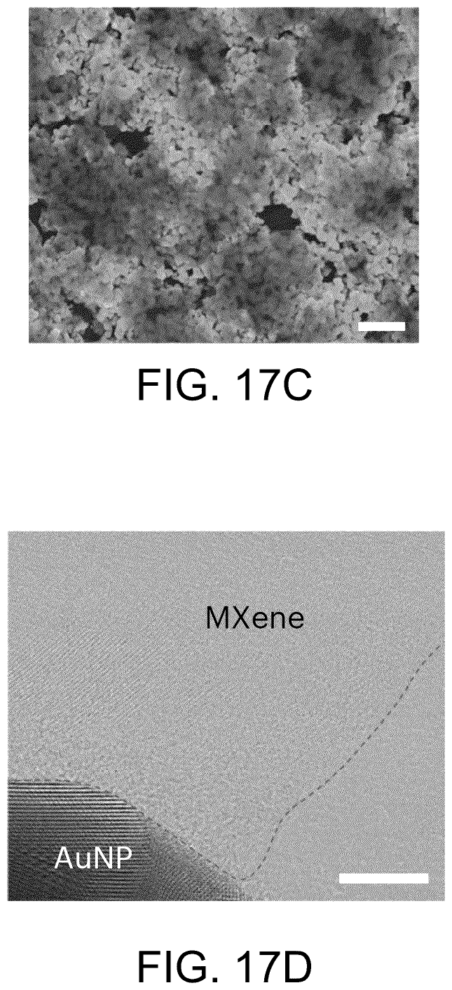

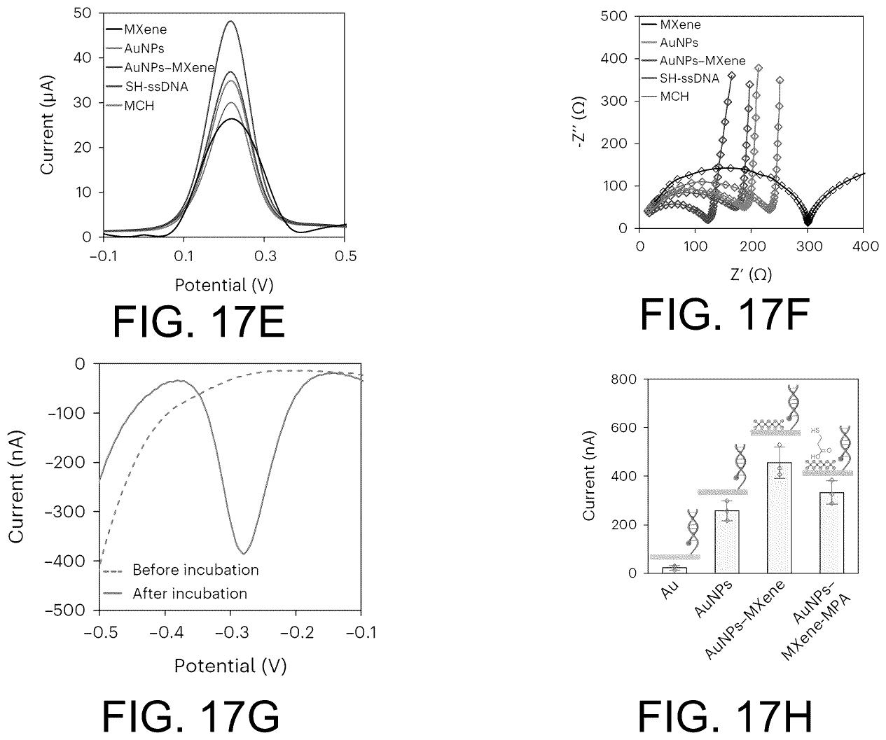

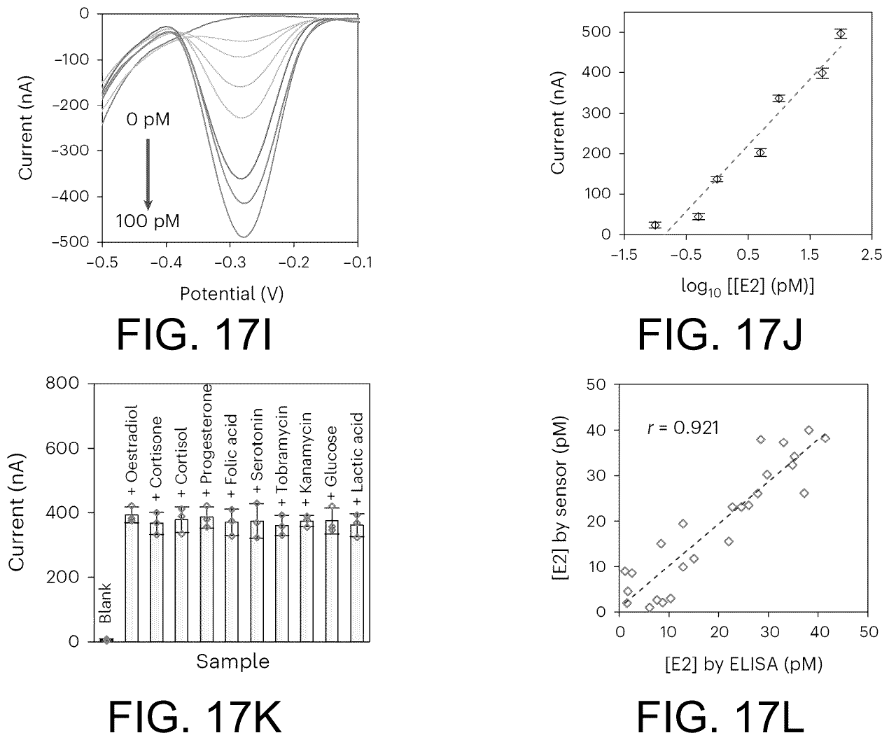

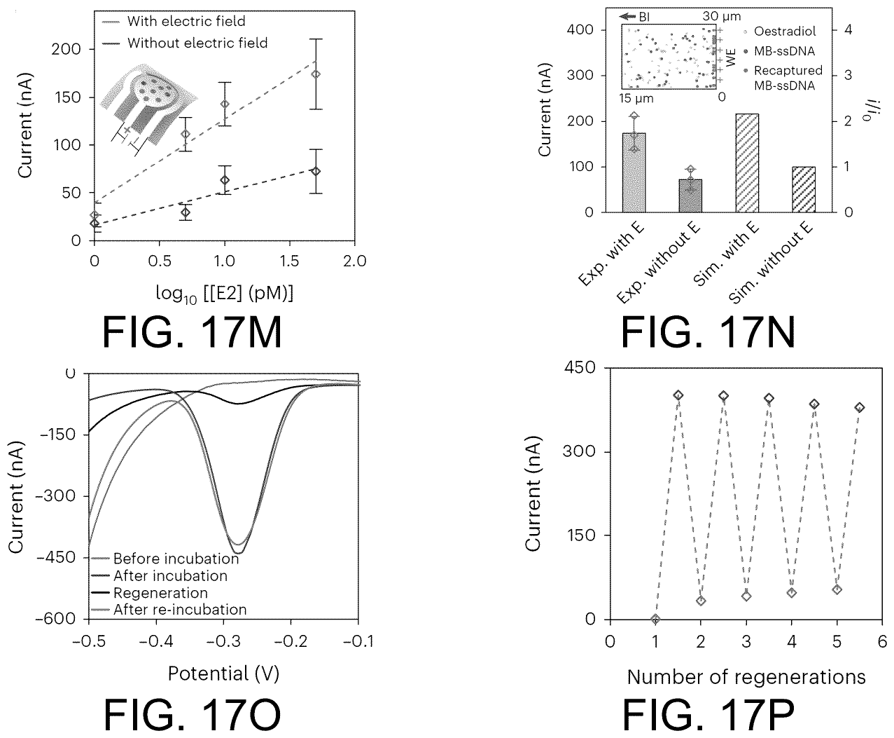

In accordance with an embodiment, the biorecognition interface and working electrode were assembled face-to-face on the sensing patch. FIG. 17A shows a schematic of the surface modification procedure of the biorecognition interface. FIG. 17B shows a shows a schematic of the surface modification procedure of the working electrode interface in this embodiment. As depicted by FIGS. 17A-17B, SH-ssDNA and aptamer-ssDNA were immobilized onto inkjet-printed AuNPs-MXene (working electrode) and AuNPs (biorecognition interface) via Au-SH binding. MCH was used to block the unbound active sites of AuNPs, subsequently, followed by rinsing with deionized water to wash away the non-electrostatically adsorbed ssDNA and aptamer-ssDNA. The redox probe MB-ssDNA was attached to the biorecognition interface to form a partially hybridized duplex with an aptamer-ssDNA, which acted as a competitive redox probe (FIGS. 15-16). Considering that a short MB-ssDNA with weak but selective affinity reaction to the estradiol aptamer was desired in this embodiment for efficient target-induced strand-displacement reaction, a 25-base MB-ssDNA (5′-GCGTGTAATTCAATAAGCTGGAAGC-3′ (SEQ ID NO: 1)) was selected on the basis of secondary structure analysis of the estradiol aptamer. A substantial hybridization reaction was obtained from the 25-base MB-ssDNA but not from the molecules with 24 or fewer bases. As depicted by FIG. 15, in the presence of estradiol, the MB-ssDNA is released by the aptamer-ssDNA because of the higher affinity of the aptamer to estradiol than the partially hybridized sequence, and then anneals to the complementary sequence at the working electrode. The recaptured MB-ssDNA probe molecules were quantified by the redox signal measured electrochemically via a SWV. It was observed that the reagentless ‘signal-on’ detection approach, coupled with highly sensitive low-background SWV measurements, offered extraordinary sensitivity and applicability for ultra-low-level sweat estradiol analysis in situ.

In this embodiment, the biorecognition interface and the detection working electrode were prepared by scalable inkjet printing of AuNPs (roughly 22 nm), which offered a high electrochemical active surface area (ECSA) for subsequent nucleic acid modifications. For example, FIG. 17C shows a SEM image of an inkjet-printed AuNPs-MXene electrode, in accordance with some implementations of the disclosure. The images are representative of three independent experiments, and the scale bar is 200 nm. On the working electrode, a thin layer of MXene, was inkjet-printed on the surface of AuNPs to further improve the sensor performance. For example, FIG. 17D shows a transmission electron microscopy characterization of AuNP-MXene, in accordance with some implementations of the disclosure. The images are representative of three independent experiments, and the scale bar is 5 nm. The MXene nanosheets used in the ink in this embodiment were largely single or few-layered, and the sheet size was smaller than 0.45 μm. SEM characterizations and experimentally obtained double layer capacitance (linearly correlated with ECSA) confirmed higher ECSA of the AuNPs-MXene electrode than those of evaporated Au and printed AuNPs electrodes. It was observed that compared to traditional screen printing, inkjet printing had multiple advantages in this embodiment, including lower cost due to less ink consumption, lower risk for sample contamination, more precise spatial and thickness control of the printed nanomaterials and higher electroactive surface area due to the porous structures to realize optimal estradiol sensing.

DPV and open circuit potential-electrochemical impedance spectroscopy (OCP-EIS) were used to further characterize the AuNPs-MXene electrode surface after each modification step (FIGS. 17E-17F, where Z, Z′ and Z″ represent impedance, resistance and reactance, respectively). The increased peak current height in DPV voltammograms and the decreased resistance in Nyquist plots after MXene printing indicated the increased electrode conductivity and electrochemical catalytic activities. The following increased resistance in Nyquist plots and decreased DPV peak current height and after SH-ssDNA and MCH modification indicated that the self-assembled monolayers and the SH-ssDNA molecules impeded the charge transfer at the electrode-solution interface due to the increased surface coverage by non-conductive molecules. It should be noted that despite MXene's high conductivity, the bare inkjet-printed MXene electrode (without AuNPs support) showed large impedance due to the lack of percolating pathway in the ultrathin film of MXene flakes. After incubation with 50 pM of estradiol, a clear methylene blue redox peak appeared at roughly −0.28 V, demonstrating the successful capture of MB-ssDNA released from the biorecognition interface due to estradiol-aptamer interaction (FIG. 17G). The incubation time, reagent concentration and measurement frequency were further optimized to reach the optimal sensor performance with high sensitivity and repeatability.

Studies showed that both AuNPs and MXene improved sensor performance (FIG. 17H; error bars represent the standard deviation of the mean from three sensors). Compared to the plain Au electrode, the AuNPs coating increased the effective electrode area, thus increasing the SH-ssDNA loading capacity for improved sensitivity. A further 40% signal enhancement was obtained with the additional MXene layer. This may be explained by MXene's ultra-high electrical conductivity, which facilitates the electrode conductivity and the efficiency of charge transfer and transport for the reduction of methylene blue molecules. The benefit of MXene was further validated by introducing 2-mercaptopropionic acid (MPA) to block the surface of MXene via the esterification interactions between the —COOH groups in the MPA and the —OH groups in the MXene. The resultant AuNPs-MXene-MPA electrode showed a substantially decreased signal, indicating the role of the MXene layer for highly sensitive estradiol analysis (FIG. 17H). It should be noted that the possible methylene blue reduction caused by MXene did not affect the final SWV estradiol quantification as the initial phase of the negative SWV scan transformed methylene blue to the oxidized state; highly stable SWV measurements were observed on MB-ssDNA and SH-ssDNA-modified AuNPs-MXene electrodes over 5,000 repetitive scans and after 0-120 minutes of solution conditioning. The estradiol sensors also displayed high operation stability in solution and high dry-state long-term storage stability under cold and room temperature conditions. The sensors from five different batches displayed high repeatability with a low coefficient of variation of 0.013.

The sensor performance was evaluated by SWV in artificial sweat (0.2×PBS, pH 7.4) containing physiologically relevant estradiol levels (0.1-100 pM) (FIG. 17I). The sensor exhibited a log-linear relationship between peak current density height of the SWV voltammograms and target concentrations with an ultra-low limit of detection of 0.14 pM (FIG. 17J). The estradiol sensor demonstrated high selectivity to estradiol over a variety of potential interferences present at much higher concentrations (FIG. 17K; error bars represent the standard deviation of the mean from three sensors). The accuracy of the aptamer sensors for sweat estradiol analysis was validated by the ELISA using iontophoresis-induced sweat samples (FIG. 17L; n=25, dashed line represents linear-fit trendline), and a high linear correlation coefficient of 0.921 between the ELISA and biosensor results was obtained.

In some embodiments where the optimal incubation time for the competitive interaction and recapture was found to be over 60 minutes, the use of an external electric field (e.g., a positive potential bias between the working and counter electrodes before incubation) was explored to reduce the incubation periods for rapid estradiol analysis. Since ssDNA is negatively charged, applying a positive potential at the working electrode can result in electrophoresis-based enhanced transport of the released MB-ssDNA redox probe across the sensing gap and substantial reduction of the necessary incubation time. As a result, electrochemical measurements revealed a substantially enhanced sensor signal after a 10 min incubation period with the application of a bias potential (1 min duration at +0.5 V before incubation) (FIG. 17M), in agreement with numerical simulation results (FIG. 17N; Exp., experimental result; Sim., simulation result; E, electric field; BI, biorecognition interface; error bars represent the standard deviation of the mean from three sensors). It was measured that the applied bias potential leads to roughly 300 nA of current flowing through the electrodes. The power dissipation due to the low current flow was at the nW level. It should be noted that the hybridized MB-ssDNA dominated the signal of the final measurement while electrostatic adsorption or chelation of MB-ssDNA onto the positively charged detection working electrode only contributed to a small portion of the final measurement result. Considering that all free MB-ssDNA molecules resulted from the selective estradiol-induced strand-displacement reaction, such electrostatic adsorption only contributed positively to sensitivity without compromising sensor selectivity.

It was observed that the estradiol sensor could be readily regenerated in deionized water (FIG. 17O) or in acidic conditions after 1 minute of incubation to perform repetitive estradiol quantification (FIG. 17P). Successful estradiol sensing and sensor regeneration showed only 6.2% signal drift after five cycles of repetitive measurement and/or regeneration (FIG. 17P; error bars represent the standard deviation of the mean from three sensors).

### Automatic Microfluidic Estradiol Analysis

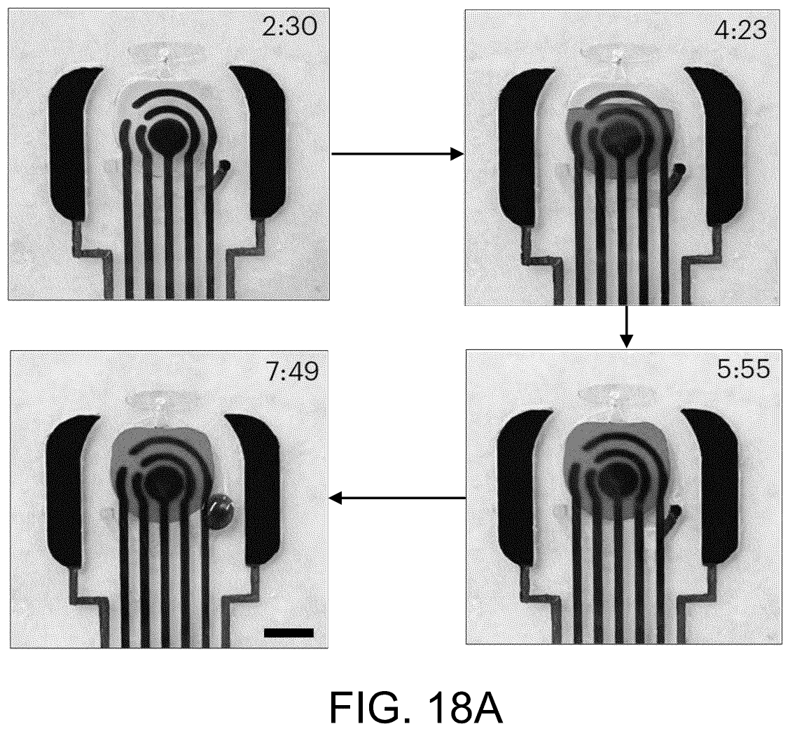

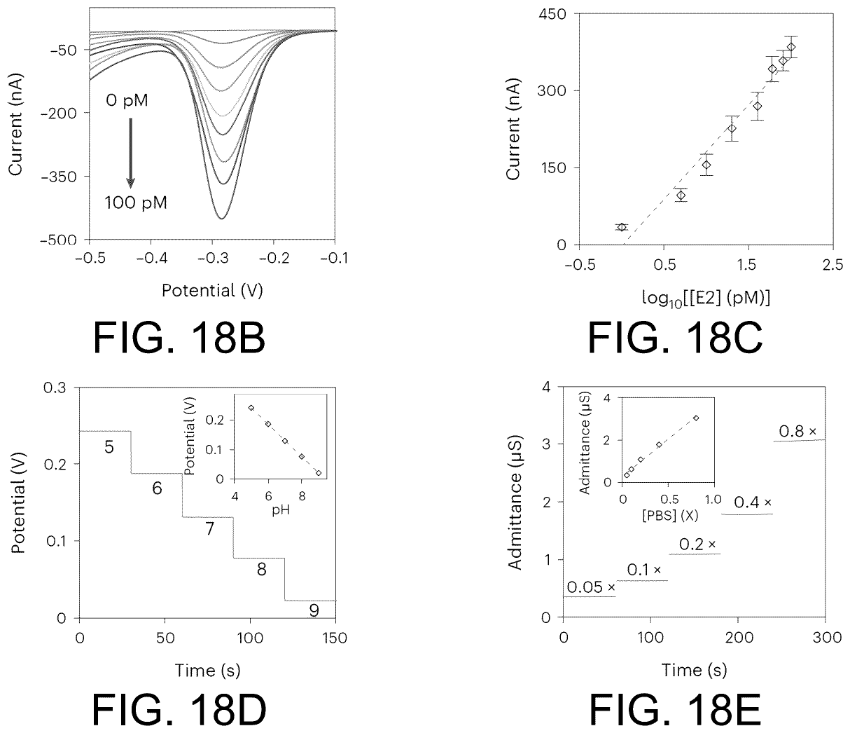

For automatic sweat estradiol analysis in situ, a disposable sensor patch was designed that consisted of a pair of carbachol gel-loaded inkjet-printed carbon electrodes for iontophoresis-based autonomous sweat induction, a microfluidic module coupled with one collection reservoir and two CBVs to precisely control the sweat sampling, and a multiplexed inkjet-printed sensor array in the microfluidic sensing reservoir for estradiol quantification and calibration. As depicted by FIG. 18A (scale bar 2.5 mm), the design was such that the induced sweat entered the microfluidic channel via the inlet, traveled into and filled the sensing reservoir (with a sample volume of roughly 6.66 μl); when the sensing reservoir was filled, estradiol analysis would initiate while newly secreted sweat was rerouted to the outlet due to capillary effects of the CBVs. Such microfluidic valving design allowed highly stable biosensing in the quiescent sweat matrix free from the influence of sweat flow. Compared to the bulk solution analysis, no apparent decrease in the sensitivity and selectivity of the estradiol sensors was observed when operating in the microfluidics (FIGS. 18B-C; error bars represent the standard deviation of the mean from three sensors).

Considering that the large interindividual variability in sweat compositions (that is, pH and ionic strength) could have a major influence on the target recognition and electrochemical measurement, a polyaniline-based potentiometric pH sensor and an impedimetric ionic strength sensor were developed and integrated into the sensor patch. In vitro sensor evaluation revealed linear relationships between the measured potential and pH for the pH sensor, and between measured admittance and electrolyte levels for the ionic strength sensor (FIGS. 18D-E). The accuracy of the pH sensor for sweat sample analysis was validated with a pH meter.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A wearable biosensor device, comprising:
a microfluidic module configured to collect a sweat sample from skin of a user, route the sweat sample to a sensing reservoir, and route additional sweat away from the sensing reservoir when the sensing reservoir is filled; and
a sensor assembly configured to quantify a biomarker contained in the sweat sample in the sensing reservoir to determine a concentration of the biomarker present in the sweat sample, the sensor assembly comprising a biorecognition interface having a surface functionalized with an aptamer that binds to the biomarker.

2. The wearable biosensor device of claim 1, wherein the microfluidic module comprises:
an inlet for collecting the sweat sample;
the sensing reservoir;
an outlet for providing an outflow of the additional sweat, the outlet fluidically positioned between the inlet and the sensing reservoir; and
a first microvalve fluidically positioned between the inlet and the outlet, the first microvalve configured to change from a closed state to an open state after the sensing reservoir is filled.

3. The wearable biosensor device of claim 2, wherein the first microvalve is a first capillary bursting valve (CBV), the first CBV having a burst pressure (BP) that is configured to be exceeded after the sensing reservoir is filled.

4. The wearable biosensor device of claim 3, wherein:
the microfluidic module further comprises a second CBV, the second CBV fluidically positioned after the sensing reservoir; and
the second CBV is configured to be in a closed state after the sensing reservoir is filled.

5. The wearable biosensor device of claim 1, wherein:
the sensor assembly further comprises a working electrode;
the surface of the biorecognition interface is further functionalized with a labeled molecule containing an electroactive label;
in response to the aptamer binding to the biomarker, the biorecognition interface is configured to release the labeled molecule; and
a surface of the working electrode is functionalized to bind to the labeled molecule released from the biorecognition interface.

6. The wearable biosensor device of claim 5, wherein:
the aptamer comprises first single-stranded deoxyribonucleic acid (ssDNA) selective to the biomarker;
the labeled molecule comprises second ssDNA; and
the surface of the working electrode is functionalized with third ssDNA configured to hybridize to the second ssDNA.

7. The wearable biosensor device of claim 6, wherein:
the first ssDNA is hybridized to the second ssDNA as a partially hybridized sequence; and
in a presence of the biomarker, the first ssDNA is configured to release the second ssDNA from the surface of the biorecognition interface due to a higher affinity of the first ssDNA to the biomarker than the partially hybridized sequence.

8. The wearable biosensor device of claim 5, wherein:
the sensor assembly further comprises a counter electrode; and
the wearable biosensor device is configured to apply an electric field between the counter electrode and the working electrode to promote transport of the labeled molecule to the working electrode.

9. The wearable biosensor device of claim 8, further comprising a flexible printed circuit board (FPCB) electrically coupled to the sensor assembly, the FPCB configured to apply the electric field as a bias potential between the counter electrode and the working electrode.

10. The wearable biosensor device of claim 5, wherein the surface of the working electrode comprises:
one or more layers of gold nanoparticles (AuNPs); and
one or more layers of MXene formed over the one or more layers of AuNPs.

11. The wearable biosensor device of claim 5, wherein:
the biomarker is a reproductive hormone; and
the sensor assembly is configured to quantify the reproductive hormone to determine the concentration of the reproductive hormone with a sensitivity of 1 picomole or less.

12. The wearable biosensor device of claim 5, wherein:
the surface of the biorecognition interface faces the surface of the working electrode;
the surface of the biorecognition interface forms a first wall of the sensing reservoir; and
the surface of the working electrode forms a second wall of the sensing reservoir, opposite the first wall.

13. The wearable biosensor device of claim 5, wherein the sensor assembly further comprises:
an ionic strength sensor configured to measure an ionic strength of the sweat sample; and
a pH sensor configured to measure a pH level of the sweat sample, wherein the wearable biosensor device is configured to calibrate readings from the working electrode based on measurements made by the ionic strength sensor and the pH sensor.

14. The wearable biosensor device of claim 1, wherein the wearable biosensor device comprises:
a disposable patch including an iontophoresis module that stimulates production of the sweat sample, the microfluidic module, and the sensor assembly, the disposable patch comprising an adhesive to directly adhere the disposable patch to the skin; and
a FPCB coupled to the disposable patch, the FPCB configured to receive signals from the sensor assembly and power the wearable biosensor device, wherein the FPCB is configured to be worn around a finger of the user.

15. The wearable biosensor device of claim 1, wherein:
the biomarker is a first type of biomarker;
the sweat sample further comprises a second type of biomarker different from the first type of biomarker;
the surface of the biorecognition interface is further functionalized with a second aptamer that binds to the second type of biomarker; and
the sensor assembly is further configured to quantify the second type of biomarker of the sweat sample in the sensing reservoir to determine a concentration of the second type of biomarker present in the sweat sample.

16. The wearable biosensor device of claim 15, wherein:
the first type of biomarker is a first type of female reproductive hormone; and
the second type of biomarker is a second type of female reproductive hormone.

17. A method, comprising:
receiving, via an inlet of a microfluidic module of a wearable biosensor device, a sweat sample collected from skin, the sweat sample including a biomarker;
collecting, within a sensing reservoir of the microfluidic module, the sweat sample, the microfluidic module comprising an outlet fluidically positioned between the inlet and the sensing reservoir, and a microvalve fluidically positioned between the inlet and the outlet, the microvalve configured to change from a closed state to an open state after the sensing reservoir is filled;
when the sensing reservoir is filled with the sweat sample, releasing, via the outlet, an additional sweat sample received by the microfluidic module via the inlet; and
estimating, using an aptamer sensor assembly of the wearable biosensor device, a concentration of the biomarker in the sweat sample collected in the sensing reservoir.

18. The method of claim 17, further comprising: regenerating the aptamer sensor assembly by rinsing the microfluidic module with deionized water or a solution with low ionic strength or acidic pH.

19. The method of claim 17, where estimating the concentration of the biomarker in the sweat sample collected in the sensing reservoir comprises:
binding, within the sensing reservoir, the biomarker of the sweat sample to an aptamer on a surface of a biorecognition interface of the aptamer sensor assembly;
in response to binding the biomarker to the aptamer, releasing, from the surface of the biorecognition interface, a molecule containing an electroactive label;
collecting, at a surface of a working electrode of the aptamer sensor assembly, the molecule released from the surface of the biorecognition interface; and
measuring an amount of electroactive label present at the surface of the working electrode to estimate a concentration of the biomarker present in the sweat sample.

20. The method of claim 19, wherein estimating the concentration of the biomarker in the sweat sample collected in the sensing reservoir, further comprises: applying an electric field between a counter electrode of the aptamer sensor assembly and the working electrode to promote transport to the working electrode of the molecule released from the surface of the biorecognition interface.

21. A method, comprising:
adhering, to skin of a user, a patch that includes a microfluidic module and sensor assembly;
collecting, in a sensing reservoir of the microfluidic module, a sweat sample obtained from the skin, the sweat sample including a reproductive hormone biomarker; and
automatically estimating, using an aptamer sensor assembly in the sensing reservoir, a concentration of the reproductive hormone biomarker in the sweat sample.

22. The method of claim 21, further comprising: presenting to the user, in real-time, via a mobile device communicatively coupled to the patch via a wireless communication medium, the concentration of the reproductive hormone biomarker estimated using the aptamer sensor assembly.


SEQUENCE LISTING (4 sequences)

SEQ ID NO: 1 - DNA, 25 bp, synthetic construct
  source 1..25: mol_type = other DNA
gcgtgtaattcaataagctggaagc

SEQ ID NO: 2 - DNA, 76 bp, synthetic construct
  source 1..76: mol_type = other DNA
  modified_base 1: mod_base = OTHER; note = Thiol Modifier C6 disulfide modified nucleotide
gcttccagcttattgaattacacgcagagggtagcggctctgcgcattcaattgctgcgcgctgaagcgcggaagc

SEQ ID NO: 3 - DNA, 25 bp, synthetic construct
  source 1..25: mol_type = other DNA
  modified_base 1: mod_base = OTHER; note = Thiol Modifier C6 disulfide modified nucleotide
gcttccagcttattgaattacacgc

SEQ ID NO: 4 - DNA, 25 bp, synthetic construct
  source 1..25: mol_type = other DNA
  modified_base 25: mod_base = OTHER; note = Methylene Blue modified nucleotide
gcgtgtaattcaataagctggaagc